(19) United States
(12) Patent Application Publication
(10) Pub. No.: US 2006/0148805 A1
(43) Pub. Date: Jul. 6, 2006

(54) Opthalmic compositions for treating ocular hypertension

(76) Inventors: Meng Hsin Chen, Westfield, NJ (US); James B. Doherty, Montvale, NJ (US); Luping Liu, Plainsboro, NJ (US); Swaminathan R. Natarajan, Scotch Plains, NJ (US); Dong-Ming Shen, Edison, NJ (US); Min Shu, Greenbrook, NJ (US)

Correspondence Address: MERCK AND CO., INC, P O BOX 2000, RAHWAY, NJ, 07065-0907, US

(21) Appl. No.: 10/561,571

(22) PCT Filed: Jun. 25, 2004
(86) PCT No.: PCT/US04/20752

§ 371 Date: Dec. 21, 2005

Related U.S. Application Data
(60) Provisional application No. 60/483,996, filed on Jul. 1, 2003.

(51) Int. Cl. A61K 31/522; A61K 31/503; A61K 31/498; A61K 31/4184; C07D 473/10; C07D 471/02; C07D 487/02

(52) U.S. Cl. 514/248; 514/249; 514/263.38; 514/303; 514/394; 544/236; 544/276; 544/330; 546/117; 548/305.4

(57) ABSTRACT

This invention relates to the use of potent potassium channel blockers or a formulation thereof in the treatment of glaucoma and other conditions which leads to elevated intraoccular pressure in the eye of a patient. This invention also relates to the use of such compounds to provide a neuroprotective effect to the eye of mammalian species, particularly humans.

### BACKGROUND OF THE INVENTION

Glaucoma is a degenerative disease of the eye wherein the intraocular pressure is too high to permit normal eye function. As a result, damage may occur to the optic nerve head and result in irreversible loss of visual function. If untreated, glaucoma may eventually lead to blindness. Ocular hypertension, i.e., the condition of elevated intraocular pressure without optic nerve head damage or characteristic glaucomatous visual field defects, is now believed by the majority of ophthalmologists to represent merely the earliest phase in the onset of glaucoma.

There are several therapies for treating glaucoma and elevated intraocular pressure, but the efficacy and the side effect profiles of these agents are not ideal. Recently potassium channel blockers were found to reduce intraocular pressure in the eye and therefore provide yet one more approach to the treatment of ocular hypertension and the degenerative ocular conditions related thereto. Blockage of potassium channels can diminish fluid secretion, and under some circumstances, increase smooth muscle contraction and would be expected to lower IOP and have neuroprotective effects in the eye. (see U.S. Pat. Nos. 5,573,758 and 5,925,342; Moore, et al., Invest. Ophthalmol. Vis. Sci 38, 1997; WO 89/10757, WO94/28900, and WO 96/33719).

### SUMMARY OF THE INVENTION

This invention relates to the use of potent potassium channel blockers or a formulation thereof in the treatment of glaucoma and other conditions that are related to elevated intraocular pressure in the eye of a patient. This invention also relates to the use of such compounds to provide a neuroprotective effect to the eye of mammalian species, particularly humans. More particularly this invention relates to the treatment of glaucoma and/or ocular hypertension (elevated intraocular pressure) using novel benzimidazole compounds having the structural formula I: 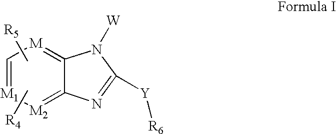
or a pharmaceutically acceptable salt, enantiomer, diastereomer or mixture thereof: wherein,
M, M1, and M2, independently are CH or N;
W represents 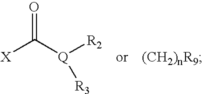
R represents hydrogen, or C₁₋₆ alkyl;
X represents —(CHR₇)ₚ—, or a bond;
Y represents —(CH₂)ᵣ—, —CO(CH₂)ₙ—, —SO₂—, —O—, —S—, —CH(OR′)—, or CONR′;
R′ represents hydrogen, C₁₋₁₀ alkyl, —(CH₂)ₙC₁₋₆ alkoxy, —(CH₂)ₙC₃₋₈ cycloalkyl, —(CH₂)ₙC₃₋₁₀ heterocyclyl, said alkyl, heterocyclyl, aryl or heteroaryl optionally substituted with 1-3 groups selected from R^{a};
or, R′ and R₆ taken together with the intervening N atom of CONR′ of Y to form a 4-10 membered carbocyclic or heterocyclic ring optionally interrupted by 1-3 atoms of O, S, C(O) or NR, and optionally having 1-4 double bonds, and optionally substituted by 1-3 groups selected from R^{a};
Q represents N, CR^{y}, or O, wherein R₂ is absent when Q is O;
R^{y} represents H, C₁₋₁₀ alkyl, C₁₋₆ alkylSR, —(CH₂)ₙO(CH₂)ₘOR,
—(CH₂)ₙC₁₋₆ alkoxy, —(CH₂)ₙC₃₋₈ cycloalkyl, —(CH₂)ₙC₃₋₁₀ heterocyclyl, —(CH₂)ₙC₅₋₁₀ heteroaryl, —N(R)₂, —COOR, or —(CH₂)ₙC₆₋₁₀ aryl, said alkyl, heterocyclyl, aryl or heteroaryl optionally substituted with 1-5 groups selected from R^{a};
or, R₂-Q-R₃ form a 3-15 membered carbocyclic or heterocyclic ring or fused ring, optionally interrupted by 1-3 atoms of O, S, C(O) or NR, and optionally having 1-5 double bonds, and optionally substituted by 1-3 groups selected from R^{a};
R_{w} represents H, C₁₋₆ alkyl, —C(O)C₁₋₆ alkyl, —C(O)OC₁₋₆ alkyl, —SO₂N(R)₂, —SO₂C₁₋₆ alkyl, —SO₂C₆₋₁₀ aryl, NO₂, CN or —C(O)N(R)₂;
R₂ represents hydrogen, C₁₋₁₀ alkyl, C₁₋₆ alkylSR, —(CH₂)ₙO(CH₂)ₘOR,
—(CH₂)ₙC₁₋₆ alkoxy, —(CH₂)ₙC₃₋₈ cycloalkyl, —(CH₂)ₙC₃₋₁₀ heterocyclyl, —(CH₂)ₙC₅₋₁₀ heteroaryl, —N(R)₂, —COOR, or —(CH₂)ₙC₆₋₁₀ aryl, said alkyl, heterocyclyl, aryl or heteroaryl optionally substituted with 1-3 groups selected from R^{a};
R₃ represents hydrogen, C₁₋₁₀ alkyl, —(CH₂)ₙC₃₋₈ cycloalkyl, —(CH₂)ₙC₃₋₁₀ heterocyclyl, —(CH₂)C₅₋₁₀ heteroaryl, —(CH₂)ₙCOOR, —(CH₂)ₙC₆₋₁₀ aryl, —(CH₂)ₙR₈, —(CH₂)ₙN(R)₂, —(CH₂)ₙNHCOOR, —(CH₂)ₙN(R₈)CO₂R, —(CH₂)ₙN(R₈)COR, —(CH₂)ₙNHCOR, (CH₂)ₙCONH(R₈), aryl, —(CH₂)ₙC₁₋₆ alkoxy, CF₃, —(CH₂)ₙSO₂R, —(CH₂)ₙSO₂N(R)₂, —(CH₂)ₙCON(R)₂, —(CH₂)ₙCONHC(R)₃, —(CH₂)ₙCOR₈, nitro, cyano or halogen, said alkyl, alkoxy, heterocyclyl, aryl or heteroaryl optionally substituted with 1-3 groups of R^{a};
R₄ and R₅ independently represent hydrogen, C₁₋₆ alkoxy, OH, OCOR₃, C₁₋₆ alkyl, COOR, SO₃H, O(CH₂)ₙN(R)₂, O(CH₂)ₙCO₂R, C₁₋₆ alkylcarbonyl, S(O)_{q}RY, (CH₂)ₙOPO(OH)₂, O(CH₂)ₙOPO(OH)₂, N(R)₂, CF₃, nitro, cyano or halogen where said alkyl, and alkoxy, are optionally substituted with 1-7 groups of R^{a};
R₆ represents hydrogen, C₁₋₁₀ alkyl, —(CH₂)ₙC₆₋₁₀ aryl, —(CH₂)ₙC₅₋₁₀ heteroaryl, (C₆₋₁₀ aryl)O—, —(CH₂)ₙC₃₋₁₀ heterocyclyl, —(CH₂)ₙC₃₋₈ cycloalkyl, —COOR, —C(O)CO₂R, said aryl, heteroaryl, heterocyclyl and alkyl optionally substituted with 1-3 groups selected from R^{a};
R₇ represents hydrogen, C₁₋₆ alkyl, —(CH₂)ₙCOOR or —(CH₂)ₙN(R)₂,
R₈ represents —(CH₂)ₙC₃₋₈ cycloalkyl, —(CH₂)_{n 3}-10 heterocyclyl, C₁₋₆ alkoxy or —(CH₂)ₙC₅₋₁₀ heteroaryl, said heterocyclyl, aryl or heteroaryl optionally substituted with 1-3 groups selected from R^{a};
R₉ represents C₁₋₁₀ alkyl, —(CH₂)ₙC₁₋₆ alkoxy, —(CH₂)ₙC₃₋₈ cycloalkyl, —(CH₂)ₙC₃₋₁₀ heterocyclyl, —(CH₂)ₙC₆₋₁₀ aryl, —(CH₂)ₙC₅₋₁₀ heteroaryl, or —N(R)₂ wherein said alkyl, alkoxy, cycloakyl, heterocyclyl, aryl, or heteroaryl are optionally substituted with 1-3 groups selected from R^{a}.
R^{a} represents P, Cl, Br, I, CF₃, N(R)₂, NO₂, CN, —COR₈, —CONHR₈, —CON(R₈)₂, —O(CH₂)ₙCOOR, —NH(CH₂)ₙOR, —COOR, —OCF₃, —NHCOR, —SO₂R, —SO₂NR₂, —SR, (C₁-C₆ alkyl)O—, —(CH₂)ₙO(CH₂)ₘOR, —(CH₂)ₙC₁₋₆ alkoxy, (aryl)O—, —OH, (C₁-C₆ alkyl)S(O)ₘ—, H₂N—C(═NH)—, (C₁-C₆ alkyl)C(O)—, (C₁-C₆ alkyl)OC(O)NH—, —(C₁-C₆ alkyl)NR_{w}(CH₂)ₙC₃₋₁₀ heterocyclyl-R_{w}, —(C₁-C₆ alkyl)O(CH₂)ₙC₃₋₁₀ heterocyclyl-R_{w}, —(C₁-C₆ alkyl)S(CH₂)ₙC₃₋₁₀ heterocyclyl-R_{w}, —C₁-C₆ alkyl)-C₃₋₁₀ heterocyclyl-R_{w}, —(CH₂)ₙ-Z¹-C(=Z²)N(R)₂, —(C₂₋₆ alkenyl)NR_{w}(CH₂)ₙC₃₋₁₀ heterocyclyl-R_{w}, —(C₂₋₆ alkenyl)O(CH₂)ₙC₃₋₁₀ heterocyclyl-R_{w}, —(C₂₋₆ alkenyl)S(CH₂)ₙC₃₋₁₀ heterocyclyl-R_{w}, —(C₂₋₆ alkenyl)-C₃₋₁₀ heterocyclyl-R—, —(C₂₋₆ alkenyl)-Z¹-C(=Z²)N(R)₂, —(CH₂)ₙSO₂R, —(CH₂)ₙSO₃H, —(CH₂)ₙPO(OR)₂, —(CH₂)ₙOPO(OR)₂, —O(CH₂)ₙSO₂R, —O(CH₂)ₙPO(OR)₂, —O(CH₂)ₙOPO(OR)₂, cyclohexyl, morpholinyl, piperidyl, pyrrolidinyl, thiophenyl, phenyl, pyridyl, imidazolyl, oxazolyl, isoxazolyl, thiazolyl, thienyl, furyl, isothiazolyl, C₂₋₆ alkenyl, and C₁-C₁₀ alkyl, said alkyl, alkenyl, alkoxy, phenyl, pyridyl, imidazolyl, oxazolyl, isoxazolyl, thiazolyl, thienyl, furyl, and isothiazolyl optionally substituted with 1-3 groups selected from C₁-C₆ alkyl, COOR, SO₃H, OH, F, Cl, Br, 1, and —O(CH₂)ₙCH(OH)CH₂SO₃H;
Z¹ and Z² independently represents NR_{w}, O, CH₂, or S;
m is 0-3;
n is 0-3;
q is 0-2;
r is 06 and
p is 0-2.

Another aspect of this invention is realized when M, M1 and M2 are all CH, and all other variables are described herein.

Another aspect of this invention is realized when at least one of M, M1 and M2 is N, and all other variables are described herein.

This and other aspects of the invention will be realized upon inspection of the invention as a whole.

### DETAILED DESCRIPTION OF THE INVENTION

The present invention is directed to novel 1,2-disubstituted benzimidazoles potassium channel blockers of Formula I. It also relates to a method for decreasing elevated intraocular pressure or treating glaucoma by administration, preferably topical or intra-camaral administration, of a composition containing a potassium channel blocker of Formula I described hereinabove and a pharmaceutically acceptable carrier.

W represents 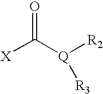
One embodiment of this invention is realized when R₃ and all other variables are as defined herein.

Another embodiment of this invention is realized when W represents (CH₂)ₙR₉ and all other variables are as defined herein.

Another embodiment of this invention is realized when X is CHR₇. Still another embodiment of this invention is realized when X is a bond. All other variables are as originally described.

Another embodiment of this invention is realized when Y is —CO(CH₂)ₙ and all other variables are as originally described. A sub-embodiment of this invention is realized when n is 0.

Another embodiment of this invention is realized when Y is CH(OR) and all other variables are as originally described.

Another embodiment of this invention is realized when Y is —(CH₂)ᵣ—

Still another embodiment of this invention is realized when Q is N and all other variables are as originally described.

Still another embodiment of this invention is realized when Q is C—Ry and all other variables are as originally described.

In another embodiment R_{w} is selected from H, C₁₋₆ alkyl, —C(O)C₁₋₆ alkyl and —C(O)N(R)₂.

Still another embodiment of this invention is realized when R₆ is C₁₋₁₀ alkyl, (CH₂)ₙC₆₋₁₀ aryl, (CH₂)ₙC₅₋₁₀ heteroaryl, (CH₂)ₙC₃₋₁₀ heterocyclyl, or (CH₂)ₙC₃₋₈ cycloalkyl, said aryl, heteroaryl, heterocyclyl and cycloalkyl optionally substituted with 1 to 3 groups of R^{a}, and all other variables are as originally described.

Yet another embodiment of this invention is realized when R₆ is (CH₂)ₙC₆₋₁₀ aryl, (CH₂)ₙC₅₋₁₀ heteroaryl or (CH₂)ₙC₃₋₁₀ heterocyclyl, said aryl, heteroaryl and heterocyclyl optionally substituted with 1 to 3 groups of R^{a}, and all other variables are as originally described.

Yet another embodiment of this invention is realized when R₇ is hydrogen Pr C₁₋₆ alkyl, and all other variables are as originally described.

Yet another embodiment of this invention is realized when Y is —CO(CH₂)ₙ, and Q is N. A subembodiment of this invention is realized when n is 0.

Still another embodiment of this invention is realized when Y is —CO(CH₂)ₙ, Q is N, R₂ and R₃ are independently selected from C₁₋₁₀ alkyl, (CH₂)ₙC₃₋₈ cycloalkyl, —(CH₂)ₙ-5˜10-membered heteroaryl, —(CH₂)ₙC₆₋₁₀ aryl, —(CH₂)ₙ-3˜10-membered heterocyclyl, and C₁₋₆ alkylOH said cycloalkyl, aryl, heteroaryl, heterocyclyl and alkyl optionally substituted with 1 to 3 groups of R^{a}.

Still another embodiment of this invention is realized when R₂ and R₃ are taken together with the intervening N atom of Q to form a 4-10 membered heterocyclic carbon ring optionally interrupted by 1-2 atoms of O, S, C(O) or NR, and optionally having 1-4 double bonds, and optionally substituted by 1-3 groups selected from R^{a};

Still another embodiment of this invention is realized when R′ and R₆ are taken together with the intervening N atom of CONR′ of Y to form a 4-10 membered carbocyclic or heterocyclic ring optionally interrupted by 1-3 atoms of O, S, C(O) or NR, and optionally having 1-5 double bonds, and optionally substituted by 1-7 groups selected from R^{a};

Another embodiment of the instant invention is realized when R^{a} is selected from F, Cl, Br, I, CF₃, N(R)₂, NO₂, CN, —CONHR₈, —CON(R₈)₂, —O(CH₂)ₙCOOR, —NH(CH₂)ₙOR, —COOR, —OCF₃, —NHCOR, —SO₂R, —SO₂NR₂, —SR, (C₁-C₆ alkyl)O—, —(CH₂)ₙO(CH₂)ₘOR, —(CH₂)ₙC₁₋₆ alkoxy, (aryl)O—, —OH, (C₁-C₆ alkyl)S(O)ₘ—, H₂N—(NH)—, (C₁-C₆ alkyl)C(O)—, (C₁-C₆ alkyl)OC(O)NH—, —(C₁-C₆ alkyl)NR_{w}(CH₂)ₙC₃₋₁₀ heterocyclyl-R_{w}, —(CH₂)ₙ-Z¹-C(=Z²)N(R)₂, —(C₂₋₆ alkenyl)NR_{w}(CH₂)ₙC₃₋₁₀ heterocyclyl-R_{w}, —(C₂₋₆ alkenyl)-Z¹-C(=Z²)N(R)₂, —(CH₂)ₙSO₂R, —(CH₂)ₙSO₃H, —(CH₂)ₙPO(OR)₂, C₂₋₆ alkenyl, and C₁-C₁₀ alkyl, said alkyl and alkenyl, optionally substituted with 1-3 groups selected from C₁-C₆ alkyl, and COOR;

Compounds and intermediates to be used in this invention are:
1-(1-Benzyl-6-methoxy-1H-benzimidazol-2-yl)-2,2-dimethylpropan-1-one,
1-(1-benzyl-5-methoxy-1H-benzimidazol-2-yl)-2,2-dimethylpropan-1-one,
1-(5-Methoxy-1H-benzimidazol-2-yl)-2,2-dimethylpropan-1-one,
Methyl[2-(2,2-dimethylpropanoyl)-6-methoxy-1H-benzimidazol-1-yl]acetate,
Methyl[2-(2,2-dimethylpropanoyl)-5-methoxy-1H-benzimidazol-1-yl]acetate,
[2-(2,2-Dimethylpropanoyl)-5-methoxy-1H-benzimidazol-1-yl]acetic acid,
2-[2-(2,2-Dimethylpropanoyl)-5-methoxy-1H-benzimidazol-1-yl]-N,N-bis(3-methylbutyl)acetamide,
1-(Diethoxymethyl)-6-methoxy-1H-benzimidazole,
1-(diethoxymethyl)-5-methoxy-1H-benzimidazole,
1-(6-Methoxy-1H-benzimidazol-2-yl)-2,2-dimethylpropan-1-one,
N,N-Dibutyl-2-[2-(2,2-dimethylpropanoyl)-5-methoxy-1H-benzimidazol-1-yl]acetamide,
2-[2-(2,2-Dimethylpropanoyl)-5-methoxy-1H-benzimidazol-1-yl)-N,N-diisobutylacetamide,
2-(2-2,2-Dimethylpropanoyl)-5-methoxy-1H-benzimidazol-1-yl]-N,N-dipropylacetamide,
N-(Cyclopropylmethyl)-2-[2-(2,2-ethylpropanoyl)-5-methoxy-1H-benzimidazol-1-yl]-N-propylacetamide,
2-[2-(2,2-Dimethylpropanoyl)-5-methoxy-1H-benzimidazol-1-yl]-N-ethyl-N-(3-methylbutyl)acetamide,
N-Butyl-2-[2-(2,2-dimethylpropanoyl)-5-methoxy-1H-benzimidazol-1-yl]-N-ethylacetamide,
N-Cyclohexyl-2-[2-(2,2-dimethylpropanoyl)-5-methoxy-1H-benzimidazol-1-yl]-N-ethylacetamide,
2-[2-(2,2-Dimethylpropanoyl)-5-methoxy-1H-benzimidazol-1-yl]-N-ethyl-N-1,3-thiazol-2-ylacetamide,
[2-(2,2-Dimethylpropanoyl)-6-methoxy-1H-benzimidazol-1-yl]acetic acid,
2-[2-(2,2-Dimethylpropanoyl)-6-methoxy-1H-benzimidazol-1-yl]-N,N-bis(3-methylbutyl)acetamide,
N,N-Dibutyl-2-[2-(2,2-dimethylpropanloyl)-6-methoxy-1H-benzimidazol-1-yl]acetamide,
2-[2-(2,2-Dimethylpropanoyl)-6-methoxy-1H-benzimidazol-1-yl]-N,N-diisobutylacetamide,
2-[2-(2,2-Dimethylpropanoyl)-6-methoxy-1H-benzimidazol-1-yl]-N,N-dipropylacetamide,
N-(Cyclopropylmethyl)-2-[2-(2,2-dimethylpropanoyl)-6-methoxy-1H-benzimidazol-1-yl]-N-propylacetamide,
2-[2-(2,2-Dimethylpropanoyl)-6-methoxy-1H-benzimidazol-1-yl]-N-ethyl-N-(3-methylbutyl)acetamide,
N-Butyl-2-[2-(2,2-dimethylpropanoyl)-6-methoxy-1H-benzimidazol-1-yl]-N-ethylacetamide,
N-Cyclohexyl-2-[2-(2,2-dimethylpropanoyl)-6-methoxy-1H-benzimidazol-1-yl]-N-ethylacetamide,
2-[2-(2,2-Dimethylpropanoyl)-6-methoxy-1H-benzimidazol-1-yl]-N-ethyl-N-1,3-thiazol-2-ylacetamide,
N-(3,3-Dimethylbutyl)-2-[2-(2,2-dimethylpropanoyl)-6-methoxy-1H-benzimidazol-1-yl]-N-ethylacetamide,
1-[2-(2,2-Dimethylpropanoyl)-5-methoxy-1H-benzimidazol-1-yl]-3,3-dimethylbutan-2-one, 1-[2-(2,2-Dimethylpropanoyl)-6-methoxy-1H-benzimidazol-1-yl]-3,3-dimethylbutan-2-one,
1-(1-Benzyl-5-methoxy-1H-benzimidazol-2-yl)-2,2-dimethylpropan-1-one,
1-(1-Benzyl-6-methoxy-1H-benzimidazol-2-yl)-2,2-dimethylpropan-1-one,
1-[1-(3,3-Dimethylbutyl)-5-methoxy-1H-benzimidazol-2-yl]-2,2-dimethylpropan-1-one,
1-[1-(3,3-Dimethylbutyl)-6-methoxy-1H-benzimidazol-2-yl]-2,2-dimethylpropan-1-one,
N,N-Dibutyl-2-[2-(2,2-dimethylpropyl)-5-methoxy-1H-benzimidazol-1-yl]acetamide,
N,N-Dibutyl-2-[2-(2,2-dimethylpropyl)-6-methoxy-1H-benzimidazol-1-yl]acetamide,
1-[2-(2,2-Dimethylpropyl)-5-methoxy-1H-benzimidazol-1-yl]-3,3-dimethylbutan-2-one,
1-[2-(2,2-Dimethylpropyl)-6-methoxy-1H-benzimidazol-1-yl]-3,3-dimethylbutan-2-one,
1-[5-Methoxy-2-(2-phenylethyl)-1H-benzimidazol-1-yl]-3,3-dimethylbutan-2-one,
1-[6-Methoxy-2-(2-phenylethyl)-1H-benzimidazol-1-yl]-3,3-dimethylbutan-2-one,
1-(5-Methoxy-2-phenyl-1H-benzimidazol-1-yl)-3,3-dimethylbutan-2-one,
1-(6-Methoxy-2-phenyl-1H-benzimidazol-1-yl)-3,3-dimethylbutan-2-one,
1-(2-Benzyl-5-methoxy-1H-benzimidazol-1-yl)-3,3-dimethylbutan-2-one,
1-(2-Benzyl-6-methoxy-1H-benzimidazol-1-yl)-3,3-dimethylbutan-2-one,
N,N-dibutyl-2-(2-isobutyryl-6-methoxy-1H-imidazo[4,5-c]pyridin-1-yl)acetamide,
N,N-dibutyl-2-(2 isobutyryl-5-methoxy-3H-imidazo[4,5-b]pyridin-3-yl)acetamide,
N,N-dibutyl-2-(2-isobutyryl-6-methoxy-1H-imidazo[4,5-b]pyridin-1-yl)acetamide,
N,N-dibutyl-2-(8-isobutyryl-2-methoxy-9H-purin-9-yl)acetamide,
N,N-dibutyl-2-(2-isobutyryl-6-methoxy-1H-imidazo[4,5-b]pyrazin-1-yl)acetamide,
N,N-dibutyl-2-(6-isobutyryl-3-methoxy-5H-imidazo[4,5-c]pyridazin-5-yl)acetamide,
N,N-dibutyl-2-(6-isobutyryl-3-methoxy-5H-imidazo[4,5-e][1,2,4]triazin-5-yl)acetamide,
N,N-dibutyl-2-[2-(2,2-dimethylpropanoyl)-5-methoxy-3H-imidazo[4,5-b]pyridin-3yl]acetamide
N,N-dibutyl-2-(2-2,2-dimethylpropanoyl)-6-methoxy-1H-imidazo[4,5-c]pyridin-1-yl)acetamide,
N,N-dibutyl-2-(2-(2,2-dimethylpropanoyl)-6-methoxy-1H-imidazo[4,5-b]pyridin-1-yl)acetamide,
N,N-dibutyl-2-(8-(2,2-dimethylpropanoyl)-2-methoxy-9H-purin-9-yl)acetamide,
N,N-dibutyl-2-(2-(2,2-dimethylpropanoyl)-6-methoxy-1H-imidazo[4,5-b]pyrazin-1-yl)acetamide,
N,N-dibutyl-2-(6-(2,2-dimethylpropanoyl)-3-methoxy-5H-imidazo[4,5-c]pyridazin-5-yl)acetamide,
N,N-dibutyl-2-(6-2,2-dimethylpropanoyl)-3-methoxy-5H-imidazo[4,5-e][1,2,4]triazin-5-yl)acetamide,
2-[2-(2,2-dimethylpropanoyl)-5-methoxy-3H-imidazo[4,5-b]pyridin-3-yl]-N,N-bis(3-methylbutyl)acetamide,
2-(2-(2,2-dimethylpropanoyl)-6-methoxy-1H-imidazo[4,5-c]pyridin-1-yl)-N,N-bis(3-methylbutyl)acetamide,
2-(2-(2,2-dimethylpropanoyl)-6-methoxy-1H-imidazo[4,5-b]pyridin-1-yl)-N,N-bis(3-methylbutyl)acetamide,
2-(8-(2,2-dimethylpropanoyl)-2-methoxy-9H-purin-9-yl)-N,N-bis(3-methylbutyl)acetamide,
2-(2-(2,2-dimethylpropanoyl)-6-methoxy-1H-imidazo[4,5-b]pyrazin-1-yl)-N,N-bis(3-methylbutyl)acetamide,
2-(6-(2,2-dimethylpropanoyl)-3-methoxy-5H-imidazo[4,5-c]pyridazin-5-yl)-N,N-bis(3-methylbutyl)acetamide,
2-[6-(2,2-dimethylpropanoyl)-3-methoxy-5H-imidazo[4,5-e][1,2,4]triazin-5-yl]-N,N-bis(3-methylbutyl)acetamide,
2-(2-isobutyryl-5-methoxy-3H-imidazo[4,5-b]pyridin-3-yl)-N,N-bis(3-methylbutyl)acetamide,
2-(2-isobutyryl-6-methoxy-1H-imidazo[4,5-c]pyridin-1-yl)-N,N-bis(3-methylbutyl)acetamide,
2-(2-isobutyryl-6-methoxy-1H-imidazo[4,5-b]pyridin-1-yl)-N,N-bis(3-methylbutyl)acetamide,
2-(8-isobutyryl-2-methoxy-9H-purin-9-yl)-N,N-bis(3-methylbutyl)acetamide,
2-(2-isobutyryl-6-methoxy-1H-imidazo[4,5-b]pyrazin-1-yl)-N,N-bis(3-methylbutyl)acetamide,
2-(6-isobutyryl-3-methoxy-5H-imidazo[4,5-c]pyridazin-5-yl)-N,N-bis(3-methylbutyl)acetamide,
2-[6-(2,2-dimethylpropanoyl)-3-methoxy-5H-imidazo[4,5-e][1,2,4]triazin-5-yl]-N,N-bis(3-methylbutyl)acetamide,
1-(2-benzoyl-6-methoxy-1H-benzimidazol-1-yl)-3,3-dimethylbutan-2-one,
2-(2-benzoyl-6-methoxy-1H-benzimidazol-1-yl)-N,N-dibutylacetamide,
2-(2-benzoyl-6-methoxy-1H-benzimidazol-1-yl)-N,N-bis(3-methylbutyl)acetamide,
2-(2-benzoyl-6-methoxy-1H-benzimidazol-1-yl)-N-butyl-N-ethylacetamide,
2-(2-benzoyl-6-methoxy-1H-benzimidazol-1-yl)-N,N-dipropylacetamide,
2-(2-benzoyl-6-methoxy-1H-benzimidazol-1-yl)-N-(tert-butyl)-N-ethylacetamide,
2-(2-benzoyl-6-methoxy-1H-benzimidazol-1-yl)-N-ethyl-N-1,3-thiazol-2-ylacetamide,
[6-methoxy-1-(3-methylbutyl)-1H-benzimidazol-2-yl](phenyl)methanone,
[1-(2-ethylbutyl)-6-methoxy-1H-benzimidazol-2-yl](phenyl)methanone,
[1-(3,3-dimethylbutyl)-6-methoxy-1H-benzimidazol-2-yl](phenyl)methanone,
N-benzyl-2-[2-(2,2-dimethylpropanoyl)-6-methoxy-1H-benzimidazol-1-yl]-N-ylacetamide,
2-(2-isobutyryl-6-methoxy-1H-benzimidazol-1-yl)-N,N-bis(3-methylbutyl)acetamide,
N,N-dibutyl-2-(2-isobutyryl-6-methoxy-1H-benzimidazol-1-yl)acetamide,
N,N-diisobutyl-2-(2-isobutyryl-6-methoxy-1H-benzimidazol-1-yl)acetamide,
2-(2-isobutyryl-6-methoxy-1H-benzimidazol-1-yl)-N,N-dipropylacetamide,
N-(cyclopropylmethyl)-2-(2-isobutyryl-6-methoxy-1H-benzimidazol-1-yl)-N-propylacetamide,
N-ethyl-2-(2-isobutyryl-6-methoxy-1H-benzimidazol-1-yl)-N-(3-methylbutyl)acetamide,
N-butyl-N-ethyl-2-(2-isobutyryl-6-methoxy-1H-benzimidazol-1-yl)acetamide,
N-cyclohexyl-N-ethyl-2-(2-isobutyryl-6-methoxy-1H-benzimidazol-yl)acetamide,
N-butyl-2-[2-(2,2-dimethylpropanoyl)-6-methoxy-1H-benzimidazol-1-yl]-N-propylacetamide,
1-(1-{2-[trans-2,5-dipropylpyrrolidin-1-yl]-2-oxoethyl}-6-methoxy-1H-benzimidazol-2-yl)-2,2-dimethylpropan-1-one,
1-(1-{2-[cis-2,5-dipropylpyrrolidin-1-yl]-2-oxoethyl}-6-methoxy-1H-benzimidazol-2-yl)-2,2-dimethylpropan-1-one,
1-(2-isobutyryl-6-methoxy-1H-benzimidazol-1-yl)-3,3-dimethylbutan-2-one,
N-(3,3-dimethylbutyl)-N-ethyl-2-(2-isobutyryl-6-methoxy-1H-benzimidazol-1-yl)acetamide,
N-butyl-2-(2-isobutyryl-6-methoxy-1H-benzimidazol-1-yl)-N-propylacetamide,
N-(3,3-dimethylbutyl)-2-[2-(2,2-dimethylpropanoyl)-6-methoxy-1H-benzimidazol-1-yl]-N-propylacetamide,
2-[2-(2,2-dimethylpropanoyl)-6-methoxy-1H-benzimidazol-1-yl]-N-(2,2-dimethylpropyl)-N-ethylacetamide,
2-{2-[4-(hydroxymethyl)benzoyl]-6-methoxy-1H-benzimidazol-1-yl}-N,N-bis(3-methylbutyl)acetamide,
2-{2-[4-(hydroxymethyl)benzoyl]-6-methoxy-1H-benzimidazol-1-yl}-N,N-diisobutylacetamide,
N-(3,3-dimethylbutyl)-N-ethyl-2-{2-[4-(hydroxymethyl)benzoyl]-6-methoxy-1H-benzimidazol-1-yl}acetamide,
2-{2-[(4-trans-hydroxycyclohexyl)carbonyl]-6-methoxy-1H-benzimidazol-1-yl}-N,N-bis(3-methylbutyl)acetamide,
N-(3,3-dimethylbutyl)-2-{2-[(4-trans-hydroxycyclohexyl)carbonyl]-6-methoxy-1H-benzimidazol-1-yl}-N-propylacetamide,
N-(3,3-dimethylbutyl)-N-ethyl-2-{2-[(4-trans-hydroxycyclohexyl)carbonyl]-6-methoxy-1H-benzimidazol-1-yl}acetamide,
N,N-bis(3,3-dimethylbutyl)-2-{2-[(4-trans-hydroxycyclohexyl)carbonyl]-6-methoxy-1H-benzimidazol-1-yl}acetamide,
2-{2-[(4-cis-hydoxycyclohexyl)carbonyl]-6-methoxy-1H-benzimidazol-1-yl}-N,N-bis(3-methylbutyl)acetamide,
2-(2-{[4-(hydroxymethyl)-1-methylcyclohexyl]carbonyl}-6-methoxy-1H-benzimidazol-1-yl)-N,N-bis(3-methylbutyl)acetamide,
N,N-dibutyl-2-(2-{[4-(hydroxymethyl)-1-methylcyclohexyl]carbonyl}-6-methoxy-1H-benzimidazol-1-yl)acetamide,
2-(2-{[4-(hydroxymethyl)-1-methylcyclohexyl]carbonyl}-6-methoxy-1H-benzimidazol-1-yl)-N,N-diisobutylacetamide,
N-(3,3-dimethylbutyl)-N-ethyl-2-(2-{[4-(hydroxymethyl)-1-methylcyclohexyl]carbonyl)-6-methoxy-1H-benzimidazol-1-yl)acetamide,
N-butyl-2-(2-([4-(hydroxymethyl)-1-methylcyclohexyl]carbonyl}-6-methoxy-1H-benzimidazol-1-yl)-N-propylacetamide,
N-(3,3-dimethylbutyl)-2-(2-{[4-(hydroxymethyl)-1-methylcyclohexyl]carbonyl}-6-methoxy-1H-benzimidazol-1-yl)-N-propylacetamide,
N-ethyl-2-(2-{[4-(hydroxymethyl)-1-methylcyclohexyl]carbonyl}-methoxy-1H-benzimidazol-1-yl)-N-(3-methylbutyl)acetamide,
1-{1-[2-(1-adamantyl)-2-oxoethyl]-6-methoxy-1H-benzimidazol-2-yl}-2,2-dimethylpropan-1-one,
1-{1-[2-(1-adamantyl)-2-oxoethyl]-6-methoxy-1H-benzimidazol-2-yl}-2-methylpropan-1-one,
1-(2-benzyl-5-methoxy-1H-benzimidazol-1-yl)-3,3-dimethylbutan-2-one,
1-(5-methoxy-2-phenyl-1H-benzimidazol-1-yl)-3,3-dimethylbutan-2-one,
1-[5-methoxy-2-(2-phenylethyl)-1H-benzimidazol-1-yl]-3,3-dimethylbutan-2-one,
   or a pharmaceutically acceptable salt, enantiomer, diastereomer or mixture thereof.

The invention is described herein in detail using the terms defined below unless otherwise specified.

The compounds of the present invention may have asymmetric centers, chiral axes and chiral planes, and occur as racemates, racemic mixtures, and as individual diastereomers, with all possible isomers, including optical isomers, being included in the present invention. (See E. L. Eliel and S. H. Wilen Stereochemistry of Carbon Compounds (John Wiley and Sons, New York 1994), in particular pages 1119-1190)

When any variable (e.g. aryl, heterocycle, R¹, R⁶ etc.) occurs more than one time in any constituent, its definition on each occurrence is independent at every other occurrence. Also, combinations of substituents/or variables are permissible only if such combinations result in stable compounds.

The term “alkyl” refers to a monovalent alkane (hydrocarbon) derived radical containing from 1 to 10 carbon atoms unless otherwise defined. It may be straight, branched or cyclic. Preferred alkyl groups include methyl, ethyl, propyl, isopropyl, butyl, t-butyl, cyclopropyl cyclopentyl and cyclohexyl. When the alkyl group is said to be substituted with an alkyl group, this is used interchangeably with “branched alkyl group”.

Cycloalkyl is a specie of alkyl containing from 3 to 15 carbon atoms, unless otherwise defined, without alternating or resonating double bonds between carbon atoms. It may contain from 1 to 4 rings, which are fused. Examples of such cycloalkyl elements include, but are not limited to, cyclopropyl, cyclobutyl, cyclopentyl, cyclohexyl and cycloheptyl.

Alkenyl is C₂-C₆ alkenyl.

Alkoxy refers to an alkyl group of indicated number of carbon atoms attached through an oxygen bridge, with the alkyl group optionally substituted as described herein. Said groups are those groups of the designated length in either a straight or branched configuration and if two or more carbon atoms in length, they may include a double or a triple bond. Exemplary of such alkoxy groups are methoxy, ethoxy, propoxy, isopropoxy, butoxy, isobutoxy, tertiary butoxy, pentoxy, isopentoxy, hexoxy, isohexoxy allyloxy, propargyloxy, and the like.

Halogen (halo) refers to chlorine, fluorine, iodine or bromine.

Aryl refers to aromatic rings e.g., phenyl, substituted phenyl and the like, as well as rings which are fused, e.g., naphthyl, phenanthrenyl and the like. An aryl group thus contains at least one ring having at least 6 atoms, with up to five such rings being present, containing up to 22 atoms therein, with alternating (resonating) double bonds between adjacent carbon atoms or suitable heteroatoms. Examples of aryl groups are phenyl, naphthyl, tetrahydronaphthyl, indanyl, biphenyl, phenanthryl, anthryl or acenaphthyl and phenanthrenyl, preferably phenyl, naphthyl or phenanthrenyl. Aryl groups may likewise be substituted as defined. Preferred substituted aryls include phenyl and naphthyl.

The term heterocyclyl or heterocyclic, as used herein, represents a stable 3- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. A fused heterocyclic ring system may include carbocyclic rings and need include only one heterocyclic ring. The term heterocycle or heterocyclic includes heteroaryl moieties. Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, dihydropyrrolyl, 1,3-dioxolanyl, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperdinyl, 2-oxopyrrolidinyl, piperidyl, piperazinyl, pyridyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, and thienyl. Preferably, heterocycle is selected from 2-azepinonyl, benzimidazolyl, 2-diazapinonyl, dihydroimidazolyl, dihydropyrrolyl, imidazolyl, 2-imidazolidinonyl, indolyl, isoquinolinyl, morpholinyl, piperidyl, piperazinyl, pyridyl, pyrrolidinyl, 2-piperidinonyl, 2-pyrimidinonyl, 2-pyrollidinonyl, quinolinyl, tetrahydrofuryl, tetrahydroisoquinolinyl, and thienyl.

The term “heteroatom” means O, S or N, selected on an independent basis.

The term “heteroaryl” refers to a monocyclic aromatic hydrocarbon group having 5 or 6 ring atoms, or a bicyclic aromatic group having 8 to 10 atoms, containing at least one heteroatom, O, S or N, in which a carbon or nitrogen atom is the point of attachment, and in which one or two additional carbon atoms is optionally replaced by a heteroatom selected from O or S, and in which from 1 to 3 additional carbon atoms are optionally replaced by nitrogen heteroatoms, said heteroaryl group being optionally substituted as described herein. Examples of such heterocyclic elements include, but are not limited to, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolyl, indolinyl, indolyl, isochromanyl, isoindolinyl, isoquinolinyl, isothiazolyl, naphthyridinyl, oxadiazolyl, pyridyl, pyrazinyl, pyrazolyl, pyridazinyl, pyrimidinyl, pyrrolyl, quinazolinyl, quinolinyl, quinoxalinyl, tetrahydroisoquinolinyl, tetrahydroquinolinyl, thiazolyl, thienofuryl, thienothienyl, thienyl and triazolyl. Additional nitrogen atoms may be present together with the first nitrogen and oxygen or sulfur, giving, e.g., thiadiazole.

This invention is also concerned with compositions and methods of treating ocular hypertension or glaucoma by administering to a patient in need thereof one of the compounds of formula I alone or in combination with one or more of the following active ingredients, in combination with a β-adrenergic blocking agent such as timolol, betaxolol, levobetaxolol, carteolol, levobunolol, a parasympathomimetic agent such as epinephrine, iopidine, brimonidine, clonidine, para-aminoclonidine, carbonic anhydrase inhibitor such as dorzolamide, acetazolamide, metazolamide or brinzolamide, an EP4 agonist (such as those disclosed in WO 02/24647, WO 02/42268, EP 1114816, WO 01/46140, PCT Appln. No. CA2004000471, and WO 01/72268), a prostaglandin such as latanoprost, travaprost, unoprostone, rescula, S1033 (compounds set forth in U.S. Pat. Nos. 5,889,052; 5,296,504; 5,422,368; and 5,151,444); a hypotensive lipid such as lumigan and the compounds set forth in U.S. Pat. No. 5,352,708; a neuroprotectant disclosed in U.S. Pat. No. 4,690,931, particularly eliprodil and R-eliprodil as set forth in WO 94/13275, including memantine; an agonist of 5-HT2 receptors as set forth in PCT/US00/31247, particularly 1-(2-aminopropyl)-3-methyl-1H-imdazol-6-ol fumarate and 2-(3-chloro-6-methoxy-indazol-1-yl)-1-methyl-ethylamine or a mixture thereof. An example of a hypotensive lipid (the carboxylic acid group on the α-chain link of the basic prostaglandin structure is replaced with electrochemically neutral substituents) is that in which the carboxylic acid group is replaced with a C₁₋₆ alkoxy group such as OCH₃ (PGF₂ₐ 1-OCH₃), or a hydroxy group (PGF₂ₐ 1-OH).

Preferred potassium channel blockers are calcium activated potassium channel blockers. More preferred potassium channel blockers are high conductance, calcium activated potassium (Maxi-K) channel blockers. Maxi-K channels are a family of ion channels that are prevalent in neuronal, smooth muscle and epithelial tissues and which are gated by membrane potential and intracellular Ca²⁺.

The present invention is based upon the finding that maxi-K channels, if blocked, inhibit aqueous humor production by inhibiting net solute and H₂O efflux and therefore lower IOP. This finding suggests that maxi-K channel blockers are useful for treating other ophthamological dysfunctions such as macular edema and macular degeneration. It is known that lowering IOP promotes blood flow to the retina and optic nerve. Accordingly, the compounds of this invention are useful for treating macular edema and/or macular degeneration.

It is believed that maxi-K channel blockers which lower IOP are useful for providing a neuroprotective effect. They are also believed to be effective for increasing retinal and optic nerve head blood velocity and increasing retinal and optic nerve oxygen by lowering IOP, which when coupled together benefits optic nerve health. As a result, this invention further relates to a method for increasing retinal and optic nerve head blood velocity, increasing retinal and optic nerve oxygen tension as well as providing a neuroprotective effect or a combination thereof.

A number of marketed drugs function as potassium channel antagonists. The most important of these include the compounds Glyburide, Glipizide and Tolbutamide. These potassium channel antagonists are useful as antidiabetic agents. The compounds of this invention may be combined with one or more of these compounds to treat diabetes.

Potassium channel antagonists are also utilized as Class 3 antiarrhythmic agents and to treat acute infarctions in humans. A number of naturally occuring toxins are known to block potassium channels including Apamin, Iberiotoxin, Charybdotoxin, Noxiustoxin, Kaliotoxin, Dendrotoxin(s), mast cell degranuating (MCD) peptide, and β-Bungarotoxin (D-BTX). The compounds of this invention may be combined with one or more of these compounds to treat arrhythmias.

Depression is related to a decrease in neurotransmitter release. Current treatments of depression include blockers of neurotransmitter uptake, and inhibitors of enzymes involved in neurotransmitter degradation which act to prolong the lifetime of neurotransmitters.

Alzheimer's disease is also characterized by a diminished neurotransmitter release. Three classes of drugs are being investigated for the treatment of Alzheimer's disease cholinergic potentiators such as the anticholinesterase drugs (e.g., physostigmine (eserine), and Tacrine (tetrahydroaminocridine)); nootropics that affect neuron metabolism with little effect elsewhere (e.g., Piracetam, Oxiracetam; and those drugs that affect brain vasculature such as a mixture of ergoloid mesylates and calcium channel blocking drugs including Nimodipine. Selegiline, a monoamine oxidase B inhibitor which increases brain dopamine and norepinephrine has reportedly caused mild improvement in some Alzheimer's patients. Aluminum chelating agents have been of interest to those who believe Alzheimer's disease is due to aluminum toxicity. Drugs that affect behavior, including neuroleptics, and anxiolytics have been employed. Anxiolytics, which are mild tranquilizers, are less effective than neuroleptics The present invention is related to novel compounds which are useful as potassium channel antagonists.

The compounds of this invention may be combined with anticholinesterase drugs such as physostigmine (eserine) and Tacrine (tetrahydroaminocridine), nootropics such as Piracetam, Oxiracetam, ergoloid mesylates, selective calcium channel blockers such as Nimodipine, or monoamine oxidase B inhibitors such as Selegiline, in the treatment of Alzheimer's disease. The compounds of this invention may also be combined with Apamin, Iberiotoxin, Charybdotoxin, Noxiustoxin, Kaliotoxin, Dendrotoxin(s), mast cell degranuating (MCD) peptide, β-Bungarotoxin (β-BTX) or a combination thereof in treating arrythmias. The compounds of this invention may further be combined with Glyburide, Glipizide, Tolbutamide or a combination thereof to treat diabetes.

The herein examples illustrate but do not limit the claimed invention. Each of the claimed compounds are potassium channel antagonists and are thus useful in the described neurological disorders in which it is desirable to maintain the cell in a depolarized state to achieve maximal neurotransmitter release. The compounds produced in the present invention are readily combined with suitable and known pharmaceutically acceptable excipients to produce compositions which may be administered to mammals, including humans, to achieve effective potassium channel blockage.

For use in medicine, the salts of the compounds of formula I will be pharmaceutically acceptable salts. Other salts may, however, be useful in the preparation of the compounds according to the invention or of their pharmaceutically acceptable salts. When the compound of the present invention is acidic, suitable “pharmaceutically acceptable salts” refers to salts prepared form pharmaceutically acceptable non-toxic bases including inorganic bases and organic bases. Salts derived from inorganic bases include aluminum, ammonium, calcium copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamin, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

The preparation of the pharmaceutically acceptable salts described above and other typical pharmaceutically acceptable salts is more fully described by Berg et al., “Pharmaceutical Salts,” J. Pharm. Sci., 1977:66:1-19.

As used herein, the term “composition” is intended to encompass a product comprising the specified ingredients in the specific amounts, as well as any product which results, directly or indirectly, from combination of the specific ingredients in the specified amounts.

When a compound according to this invention is administered into a human subject, the daily dosage will normally be determined by the prescribing physician with the dosage generally varying according to the age, weight, sex and response of the individual patient, as well as the severity of the patient's symptoms.

The maxi-K channel blockers used can be administered in a therapeutically effective amount intravaneously, subcutaneously, topically, transdermally, parenterally or any other method known to those skilled in the art.

Ophthamic pharmaceutical compositions are preferably adapted for topical administration to the eye in the form of solutions, suspensions, ointments, creams or as a solid insert. Ophthalmic formulations of this compound may contain from 0.01 ppm to 1% and especially 0.1 ppm to 1% of medicament. Higher dosages as, for example, about 10% or lower dosages can be employed provided the dose is effective in reducing intraocular pressure, treating glaucoma, increasing blood flow velocity or oxygen tension. For a single dose, from between 0.1 ng to 5000 ug, preferably 1 ng to 500 ug, and especially 10 ng to 100 ug of the compound can be applied to the human eye.

The pharmaceutical preparation which contains the compound may be conveniently admixed with a non-toxic pharmaceutical organic carrier, or with a non-toxic pharmaceutical inorganic carrier. Typical of pharmaceutically acceptable carriers are, for example, water, mixtures of water and water-miscible solvents such as lower alkanols or aralkanols, vegetable oils, polyalkylene glycols, petroleum based jelly, ethyl cellulose, ethyl oleate, carboxymethyl-cellulose, polyvinylpyrrolidone, isopropyl myristate and other conventionally employed acceptable carriers. The pharmaceutical preparation may also contain non-toxic auxiliary substances such as emulsifying, preserving, wetting agents, bodying agents and the like, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic acid, and the like. Additionally, suitable ophthalmic vehicles can be used as carrier media for the present purpose including conventional phosphate buffer vehicle systems, isotonic boric acid vehicles, isotonic sodium chloride vehicles, isotonic sodium borate vehicles and the like. The pharmaceutical preparation may also be in the form of a microparticle formulation. The pharmaceutical preparation may also be in the form of a solid insert. For example, one may use a solid water soluble polymer as the carrier for the medicament. The polymer used to form the insert may be any water soluble non-toxic polymer, for example, cellulose derivatives such as methylcellulose, sodium carboxymethyl cellulose, (hydroxyloweralkyl cellulose), hydroxyethyl cellulose, hydroxypropyl cellulose, hydroxypropylmethyl cellulose; acrylates such as polyacrylic acid salts, ethylacrylates, polyactylamides; natural products such as gelatin, alginates, pectins, tragacanth, karaya, chondrus, agar, acacia; the starch derivatives such as starch acetate, hydroxymethyl starch ethers, hydroxypropyl starch, as well as other synthetic derivatives such as polyvinyl alcohol, polyvinyl pyrrolidone, polyvinyl methyl ether, polyethylene oxide, neutralized carbopol and xanthan gum, gellan gum, and mixtures of said polymer.

Suitable subjects for the administration of the formulation of the present invention include primates, man and other animals, particularly man and domesticated animals such as cats and dogs.

The pharmaceutical preparation may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

The ophthalmic solution or suspension may be administered as often as necessary to maintain an acceptable IOP level in the eye. It is contemplated that administration to the mamalian eye will be about once or twice daily.

For topical ocular administration the novel formulations of this invention may take the form of solutions, gels, ointments, suspensions or solid inserts, formulated so that a unit dosage comprises a therapeutically effective amount of the active component or some multiple thereof in the case of a combination therapy.

The following examples given by way of illustration is demonstrative of the present invention.

The compounds of this invention can be made, with modification where appropriate, in accordance with Schemes 1-3. Examples 26-36 are also produced in accordance with Schemes 3.

One method for the preparation of compounds in the present invention is illustrated in Scheme 1.

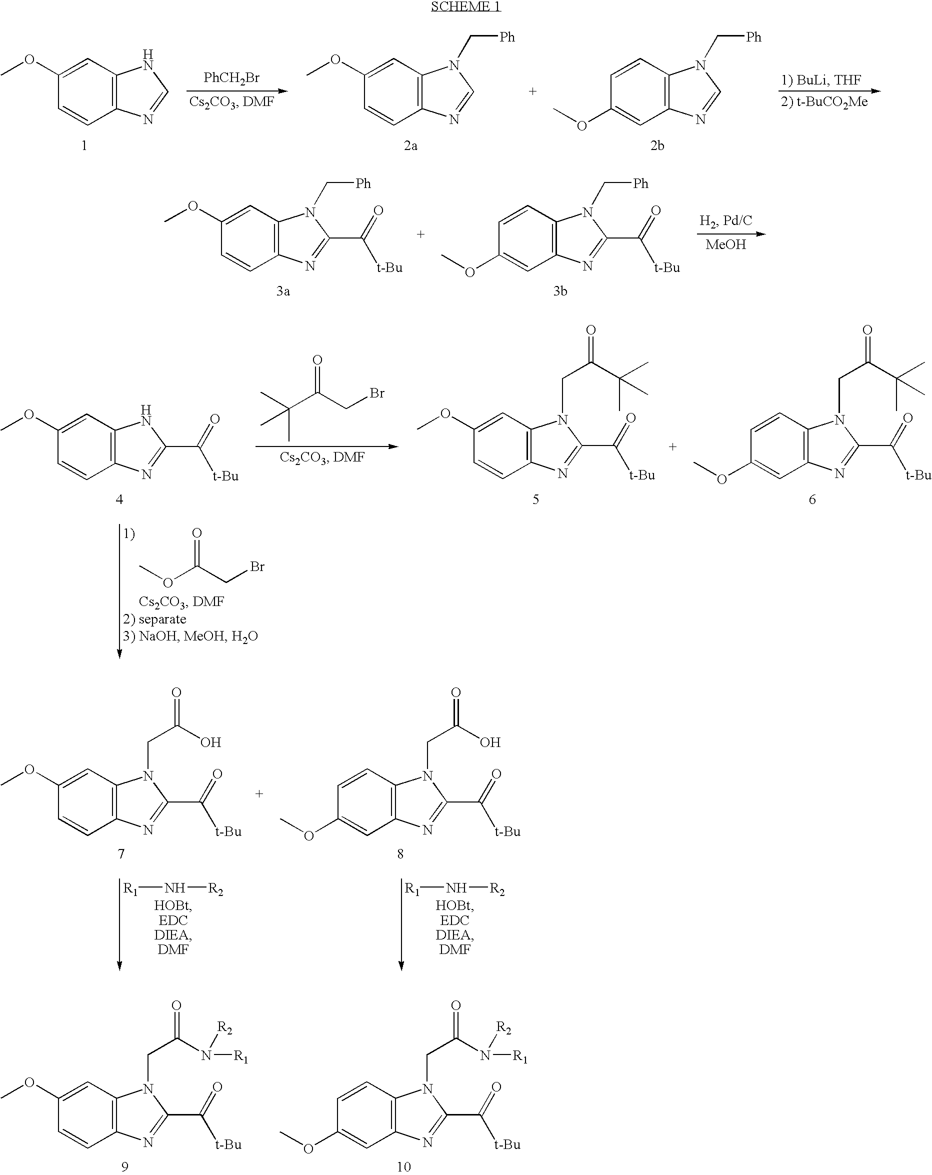

Commercially available benzimidazole 1 was protected with a benzyl group using standard conditions to give an isomeric mixture 2a and 2b. This mixture was converted to acyl compounds such as 3a and 3b using a procedure based on Carr et al. J. Org. Chem. 1990, 55, 1399. The benzyl group was removed by hydrogenolysis to give acyl compound 4. Compound 4 can be alkylated with bromoketones to give desired compounds such as 5 and 6, which can be separated. Alternatively, 4 can be alkylated with a bromoester, the ester mixture separated, and the individual ester hydrolyzed to give acid 7 and 8. These acids can be converted to amides 9 and 10 using standard conditions.

An alternative and more preferred method for the preparation of the crucial intermediate 4 is illustrated in Scheme 2. Heating 1 with excess ortho formate as solvent in the presence of catalytic amount of p-toluenesulfonic acid gave a mixture of protected benzimidazoles 11a and 11b. This mixture was treated with based and reacted with an ester. During the acidic workup, the protection group was removed to give compounds such as 4.

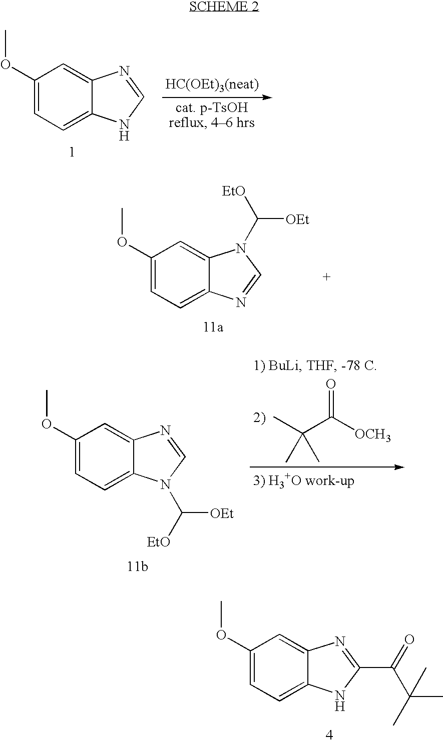

General Experimental Conditions: NMR spectra were recorded at room temperature on Varian Instruments referenced to residual solvent peak. LC-MS were measured on an Aglient HPLC and MicroMass ZQ detector with electrospray ionization using a 2.0×50 mm X-Terra C18 column and 10˜98% MeCN gradient over 3.75 minutes followed by 98% MeCN for 1 minute. The aqueous and MeCN eluents contained 0.06 and 0.05% (v/v) trifluoroacetic acid, respectively. Preparative TALC separations were done using a YMC 20×150 mm 5μ ProC18 column or a 9.4×250 mm SB-C18 Zorbax column.

### EXAMPLE 1

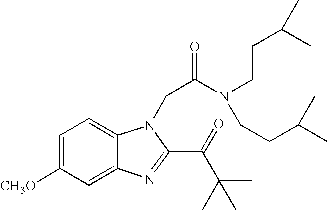

### 2-[2-(2,2-Dimethylpropanoyl)-5-methoxy-1H-benzimidazol-1-yl]-N,N-bis(3-methylbutyl)acetamide

### Step A: 1-Benzyl-6-methoxy-1H-benzimidazole and 1-benzyl-5-methoxy-1H-benzimidazole

To a mixture of 4.13 g 5-methoxy-1H-bezimidazole and 11.8 g cesium carbonate in 100 mL dimethylformamide (DMF) was added 6.3 g benzyl bromide. After stirring the mixture at room temperature for 3 days, it was quenched by addition of saturated ammonium chloride solution. It was diluted with water and extracted with ethyl acetate. The ethyl acetate solution was washed with saturated brine, dried over anhydrous Na₂SO₄ and evaporated under reduced pressure. The residue was purified on silica gel eluting with hexanes and ethyl acetate (1:3 to 1:4 v/v) followed by 1:4 hexanes and ethyl acetate with 1% methanol. The fractions containing pure products were pooled and evaporated to give the title compounds in about 1.2:1 ratio. ¹H NMR, (CDCl₃, 500 MHz) 67 Major isomer: 7.89 (s, 1H), 7.72 (d, J=8.7 Hz, 1H), 7.34˜7.39 (m, 3H), 7.20˜7.21 (m, 2H), 6.94 (dd, J=2.3 & 8.7 Hz, 1H), 6.75 (d, J=2.6 Hz, 1H), 5.34 (s, 2H), 3.82 (s, 3H); Minor isomer: 7.97 (s, 1H), 7.34˜7.39 (m, 3H), 7.33 (d, J=2.6 Hz, 1H), 7.20˜7.21 (m, 2H), 7.17 (d, J=8.9 Hz, 1H), 6.92 (dd, J=2.5 & 9.0 Hz, 1H), 5.35 (s, 2H), 3.88 (s, 3H). LC-MS: 2.08 minute (M+H=239.2).

### Step B: 1-(1-Benzyl-6-methoxy-1H-benzimidazol-2-yl)-2,2-dimethylpropan-1-one and 1-(1-benzyl-5-methoxy-1H-benzimidazol-2-yl)-2,2-dimethylpropan-1-one

To a solution of 1.24 g product from the Step A above in 13 mL anhydrous tetrahydrofuran (THF) cooled with an acetone dr ice bath at −78° C. was added 2.2 mL 2.5 M n-BuLi in hexane. The resulting red solution was stirred for 10 minutes. A solution of 0.61 g methyl pivalate in 6.5 mL anhydrous THF was added. After stirring the reaction mixture in the cooling bath for 30 minutes, the cooling bath was removed and the reaction mixture was allowed to warm to room temperature. It was quenched by addition of saturated ammonium chloride solution, diluted with water and extracted with ethyl acetate. The ethyl acetate solution was washed with saturated brine, dried over anhydrous Na₂SO₄ and evaporated under reduced pressure. The residue was purified by chromatography (silica, 5:1 hexanes and EtOAc) to give the title compounds as a mixture. LC-MS: 4.20 minute (M+H=323.4). For NMR, see Examples 22 and 23.

### Step C: 1-(5-Methoxy-1H-benzimidazol-2-yl)-2,2-dimethylpropan-1-one

A mixture of 1.23 g product from the Step B above and 0.21 g 10% Pd/C in 40 mL methanol was treated with hydrogen from a balloon over night. After purging the reaction mixture with nitrogen, it was filtered and evaporated under reduced pressure. The residue was purified by chromatography (silica, 7.5:1 to 1:1 hexanes and EtOAc) to give some recovered starting material followed by the title compound. ¹H NMR (CDCl₃, 500 MHz) δ 7.69 (br d, J=7.8 Hz, 1H), 7.10 (br s, 1H), 7.05 (dd, J=2.4 & 9.0 Hz, 1H), 3.90 (s, 3H), 1.58 (s, 9H). LC-MS: 3.25 minute (M+H=233.3).

### Step D: Methyl[2-(2,2-dimethylpropanoyl)-6-methoxy-1H-benzimidazol-1-yl acetate and methyl[2-(2,2-dimethylpropanoyl)-5-methoxy-1H-benzimidazol-1-yl]acetate

To a mixture of 0.33 g product from the Step C above and 0.61 g cesium carbonate in 10 mL dry DMF was added 0.29 g methyl bromoacetate. The mixture was heating at 40° C. over night. It was quenched by addition of saturated ammonium chloride solution, diluted with water and extracted with ethyl acetate. The ethyl acetate solution was washed with saturated brine, dried over anhydrous Na₂SO₄ and evaporated under reduced pressure. The residue was purified by chromatography (silica, 5:1 hexanes and EtOAc) to give methyl[2-2,2-dimethylpropanoyl)-5-methoxy-1H-benzimidazol-1-yl]acetate followed by methyl[2-(2,2-dimethylpropanoyl)-6-methoxy-1H-benzimidazol-1-yl]acetate. ¹H NMR (CDCl₃, 500 MHz) of methyl 12-(2,2-dimethylpropanoyl)-5-methoxy-1H-benzimidazol-1-yl]acetate & 7.35 (d, J=2.3 Hz, 1H), 7.23 (d, J=8.9 Hz, 1H), 7.10 (dd, J=2.3 & 8.9 Hz, 1H), 5.24 (s, 2H), 3.90 (s, 3H), 3.78 (s, 3H), 1.55 (s, 9H). ¹H NMR (CDCl₃, 500 MHz) of methyl[2-(2,2-dimethylpropanoyl)-6-methoxy-1H-benzimidazol-1-yl]acetate δ 7.78 (d, J=8.9 Hz, 1H), 7.02 (dd, J=2.3 & 8.9 Hz, M1), 6.70 (d, J=2.3 Hz, 1H), 5.23 (s, 2H), 3.90 (s, 3H), 3.79 (s, 3H), 1.55 (s, 9H). The isomers were identified by NOE difference spectra.

### Step E: [2-(2,2-Dimethylpropanoyl)-5-methoxy-1H-benzimidazol-1-yl]acetic acid

A solution of 0.147 g methyl[2-2,2-dimethylpropanoyl)-5-methoxy-1H-benzimidazol-1-yl]acetate from the Step D above in 5 mL methanol was treated with 0.5 mL 5 N NaOH solution at room temperature over night. The solvents were removed completely under reduced pressure. The residue was dissolved in 5 mL water and the product was precipitated by adding 2.8 mL 1 N HCl. The precipitate was collected by filtration, washed with water, and dried to give the title compound. 1H NMR (CD₃OD, 500 MHz) δ 7.44 (d, J=8.9 Hz, 1H), 7.29 (d, 2.3 Hz, 1H), 7.08 (dd, J=2.3 & 8.9 Hz, 1H), 5.25 (s, 2H), 3.87 (s, 3H), 1.49 (s, 9H). LC-MS: 3.33 minute (M+H=291.4).

### Step F: 2-[2-(2,2-Dimethylpropanoyl)-5-methoxy-1H-benzimidazol-1-yl)-N,N-bis(3-methylbutyl)acetamide

To a mixture of 9 mg [2-(2,2-dimethylpropanoyl)-5-methoxy-1H-benzimidazol-1-yl]acetic acid from the Step E above, 6.3 mg 1-hydroxybenzotriazole hydrate (HOBt), and 11.9 mg 1-(3-dimethylaminopropyl)-3-ethylcarbodiimide hydrochloride (EDC) was added 0.5 mL dry DMF, followed by 9.5 μL di-iso-amylamine and 20.0 μL di-iso-propylethylamine (DIEA). This solution was heated at 53° C. for 3 hours. It was purified directly on RP-HPLC using 70˜100% MeCN gradient. The fractions containing pure product were pooled and lyophilized to give the title compound. LC-MS: 4.53 minute (M+H=430.5).

### EXAMPLE 2

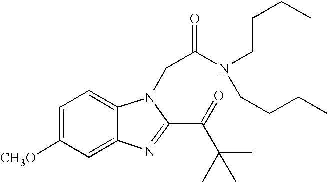

### N,N-Dibutyl-2-[2-(2,2-dimethylpropanoyl)-5-methoxy-1H-benzimidazol-1-yl]acetamide

To a mixture of 8.2 mg [2-(2,2-dimethylpropanoyl)-5-methoxy-1H-benzimidazol-1-yl]acetic acid from the Step E Example 1, 5.7 mg HOBt, and 10.8 mg EDC was added 0.5 mL dry DMF, followed by 7.2 μL di-n-butylamine and 18.2 μL DIEA. This solution was heated at 53° C. for 3 hours. It was purified directly on RP-HPLC using 65˜100% MeCN gradient. The fractions containing pure product were pooled and lyophilized to give the title compound. LC-MS: 4.30 minute (M+H=402.5).

### EXAMPLE 3

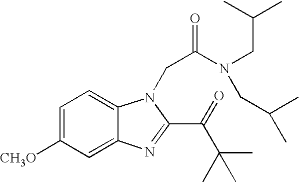

### 2-[2-(2,2-Dimethylpropanoyl)-5-methoxy-1H-benzimidazol-1-yl]-N,N-diisobutylacetamide

To a mixture of 8.9 mg [2-(2,2-dimethylpropanoyl)-5-methoxy-1H-benzimidazol-1-yl]acetic acid from the Step E Example 1, 6.2 mg HOBt, and 11.8 mg EDC was added 0.5 mL dry DMF, followed by 8.0 μL di-iso-butylamine and 19.8 μL DIEA. This solution was heated at 53° C. for 3 hours. It was purified directly on RP-HPLC using 65˜100% MeCN gradient. The fractions containing pure product were pooled and lyophilized to give the title compound. LC-MS: 4.28 minute (M+H=402.4).

### EXAMPLE 4

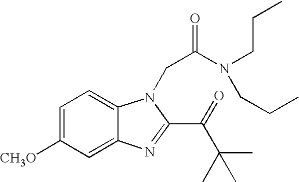

### 2-[2-(2,2-Dimethylpropanoyl)-5-methoxy-1H-benzimidazol-1-yl]-N,N-dipropylacetamide

To a mixture of 9 mg [2-(2,2-dimethylpropanoyl)-5-methoxy-1H-benzimidazol-1-yl]acetic acid from the Step E Example 1, 6.3 mg HOBt, and 11.9 mg EDC was added 0.5 mL dry DMF, followed by 7.1 μL di-n-propylamine and 20.0 μL DIEA. This solution was heated at 53° C. for 3 hours. It was purified directly on RP-HPLC using 60˜100% MeCN gradient. The fractions containing pure product were pooled and lyophilized to give the title compound. LC-MS: 3.98 minute (M+H=374.4).

### EXAMPLE 5

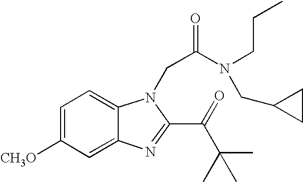

### N-(Cyclopropylmethyl)-2-[2-(2,2-dimethylpropanoyl)-5-methoxy-1H-benzimidazol-1-yl]-N-propylacetamide

To a mixture of 8.7 mg [2-(2,2-dimethylpropanoyl)-5-methoxy-1H-benzimidazol-1-yl]acetic acid from the Step E Example 1, 6.1 mg HOBt, and 11.5 mg EDC was added 0.5 mL dry DMF, followed by 8.8 μL N-propylcyclopropanemethyl-amine and 19.3 μL DIEA. This solution was heated at 53° C. for 3 hours. It was purified directly on RP-HPLC using 60˜100% MeCN gradient. The fractions containing pure product were pooled and lyophilized to give the title compound. LC-MS: 4.01 minute (M+H=386.4).

### EXAMPLE 6

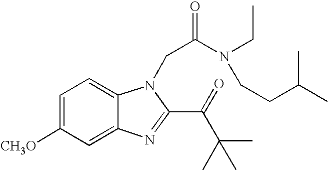

### 2-[2-(2,2-Dimethylpropanoyl)-5-methoxy-1H-benzimidazol-1-yl]-N-ethyl-N-3-methylbutyl)acetamide

To a mixture of 8.9 mg [2-(2,2-dimethylpropanoyl)-5-methoxy-1H-benzimidazol-1-yl]acetic acid from the Step E Example 1, 6.2 mg HOBt, and 11.8 mg EDC was added 0.5 mL dry DMF, followed by 7.1 μL N-ethyl-1-amylamine and 19.8 μL DIEA. This solution was heated at 53° C. for 3 hours. It was purified directly on RP-HPLC using 65˜100% MeCN gradient. The fractions containing pure product were pooled and lyophilized to give the title compound. LC-MS: 4.14 minute (M+H=388.4).

### EXAMPLE 7

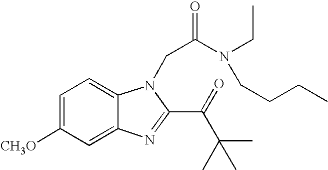

### N-Butyl-2-[2-(2,2-dimethylpropanoyl)-5-methoxy-1H-benzimidazol-1-yl]-N-ethylacetamide

To a mixture of 8.2 mg [2-(2,2-dimethylpropanoyl)-5-methoxy-1H-benzimidazol-1-yl]acetic acid from the Step E Example 1, 5.7 mg HOBt, and 10.8 mg EDC was added 0.5 mL dry DMF, followed by 5.9 μL N-ethylbutylamine and 18.2 μL DIEA. This solution was heated at 53° C. for 3 hours. It was purified directly on RP-HPLC using 60˜100% MeCN gradient. The fractions containing pure product were pooled and lyophilized to give the title compound. LC-MS: 3.99 minute (M+H=374.4).

### EXAMPLE 8

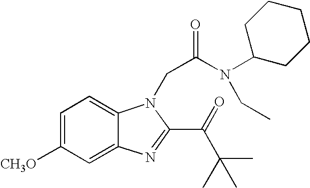

### N-Cyclohexyl-2-[2-(2,2-dimethylpropanoyl)-5-methoxy-1H-benzimidazol-1-yl]-N-ethylacetamide

To a mixture of 8.4 mg [2-(2,2-dimethylpropanoyl)-5-methoxy-1H-benzimidazol-1-yl]acetic acid from the Step E Example 1, 5.9 mg HOBt, and 11.1 mg EDC was added 0.5 mL dry DMF, followed by 6.9 μL N-ethylcyclohexylamine and 18.6 μL DIEA. This solution was heated at 53° C. for 3 hours. It was purified directly on RP-HPLC using 65˜100% MeCN gradient. The fractions containing pure product were pooled and lyophilized to give the title compound. LC-MS: 4.16 minute (M+H=400.4).

### EXAMPLE 9

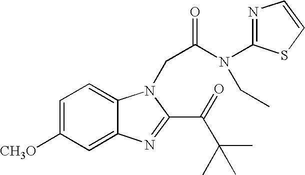

### 2-[2-(2,2-Dimethylpropanoyl)-5-methoxy-1H-benzimidazol-1-yl]-N-ethyl-N-1,3-thiazol-2-ylacetamide

### Step A: N-Ethyl-1,3-thiazol-2-amine

To a suspension of an N-ethylthiourea in ethanol was added 1,1-dimethoxy-2-bromoethane and concentrated HCl. The reaction mixture is heated at reflux to give title compound after chromatography on silica. ¹H NMR (CDCl₃, 500 MHz) δ 7.14 (d, J=3.6 Hz, 1H), 6.51 (d, J=3.7 Hz, 1H), 5.47 (v br s, 1H), 3.34 (q, J=7.2 Hz, 2H), 1.32 (t, J=7.2 Hz, 3H).

### Step B: 2-(2-(2,2-Dimethylpropanoyl)-5-methoxy-1H-benzimidazol-1-yl]-N-ethyl-N-1,3-thiazol-2-ylacetamide

To a mixture of 7.2 mg [2-(2,2-dimethylpropanoyl)-5-methoxy-1H-benzimidazol-1-yl]acetic acid from the Step E Example 1, 4.8 mg N-ethyl-1,3-thiazol-2-amine from the Step A above, 5.0 mg HOBt, and 9.5 mg EDC was added 0.5 mL dry DMF, followed by 16.0 μL DIEA. This solution was heated at 53° C. for 3 hours. It was purified directly on RP-HPLC using 55˜100% MeCN gradient. The fractions containing pure product were pooled and lyophilized to give the title compound. LC-MS: 3.92 minute (M+H=401.4).

### EXAMPLE 10

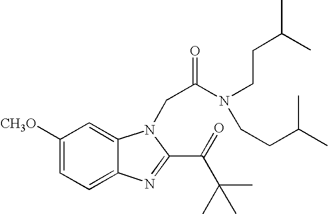

### 2-[2-(2,2-Dimethylpropanoyl)-6-methoxy-1H-benzimidazol-1-yl]-N,N-bis(3-methylbutyl)acetamide

### Step A: [2-(2,2-Dimethylpropanoyl)-6-methoxy-1H-benzimidazol-1-yl]acetic acid

A solution of 0.222 g methyl[2-(2,2-dimethylpropanoyl)-6-methoxy-1H-benzimidazol-1-yl]acetate from the Step D Example 1 in 7 mL methanol was treated with 0.7 mL 5 N NaOH solution at room temperature over night. The solvents were removed completely under reduced pressure. The residue was dissolved in 5 mL water and the product was precipitated by adding 4 mL 1 N HCl. The precipitate was collected by filtration, washed with water, and dried to give the title compound. ¹H NMR (CD₃OD, 500 MHz) δ 7.68 (d, J=9.0 Hz, 1H), 7.05 (d, 2.3 Hz, 1H), 6.99 (dd, J=2.3 & 9.0 Hz, 1H), 5.27 (s, 2H), 3.88 (s, 3H), 1.48 (s, 9H).

### Step B: 2-[2-(2,2-Dimethylpropanoyl)-6-methoxy-1H-benzimidazol-1-yl]-N,N-bis(3-methylbutyl)acetamide

To a mixture of 9.5 mg [2-(2,2-dimethylpropanoyl)-6-methoxy-1H-benzimidazol-1-yl]acetic acid from the Step A above, 6.6 HOBt, and 12.5 EDC was added 0.5 mL dry DMF, followed by 10.0 μL di-1-amylamine and 21.1 μL DIEA. This solution was heated at 40° C. over night. It was purified directly on RP-HPLC using 70˜100% MeCN gradient. The fractions containing pure product were pooled and lyophilized to give the title compound. LC-MS: 4.54 minute (M+H=430.4).

### EXAMPLE 11

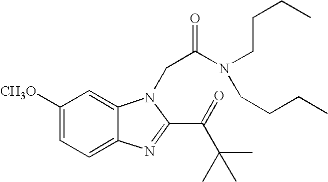

### N,N-Dibutyl-2-[2-(2,2-dimethylpropanoyl)-6-methoxy-1H-benzimidazol-1-yl]acetamide

To a mixture of 8.6 mg [2-(2,2-dimethylpropanoyl)-6-methoxy-1H-benzimidazol-1-yl]acetic acid from the Step A Example 10, 6.0 mg HOBt, and 11.4 mg EDC was added 0.5 mL dry DMF, followed by 7.5 μL di-n-butylamine and 19.1 μL DIEA. This solution was heated at 40° C. over night. It was purified directly on RP-HPLC using 65-100% MeCN gradient. The fractions containing pure product were pooled and lyophilized to give the title compound. ¹H NMR (CDCl₃, 500 MHz) δ 7.76 (d, J=8.9 Hz, 1H), 6.98 (dd, J=2.3 & 8.9 Hz, 1H), 6.69 (d, J=2.3 Hz, 1H), 5.31 (s, 2H), 3.89 (s, 3H), 3.42 (t, J=7.8 Hz, 2H), 3.36 (t, J=7.6 Hz, 2M), 1.72˜1.78 (m, 2M, 1.44˜1.59 (m, 4H), 1.53 (s, 933), 1.27˜1.35 (m, 2H), 1.06 (t, J=7.3 Hz, 3H), 0.92 (t, J=7.4 Hz, 31H). LC-MS: 4.31 minute (M+H=402.5).

### EXAMPLE 12

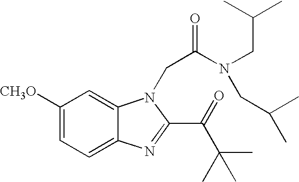

### 2-[2-(2,2-Dimethylpropanoyl)-6-methoxy-1H-benzimidazol-1-yl]-N,N-diisobutylacetamide

To a mixture of 8 mg [2-(2,2-dimethylpropanoyl)-6-methoxy-1H-benzimidazol-1-yl]acetic acid from the Step A Example 10, 5.6 mg HOBt, and 10.6 mg EDC was added 0.5 mL dry DMF, followed by 7.2 μL di-1-butylamine and 17.8 μL DIEA. This solution was heated at 40° C. over night. It was purified directly on RP-HPLC using 65˜100% MeCN gradient. The fractions containing pure product were pooled and lyophilized to give the title compound. LC-MS: 4.30 minute (M+H=402.4).

### EXAMPLE 13

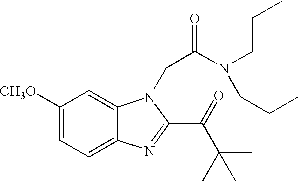

### 2-[2-(2,2-Dimethylpropanoyl)-6-methoxy-1H-benzimidazol-1-yl]-N,N-dipropylacetamide

To a mixture of 8.2 mg [2-(2,2-dimethylpropanoyl)-6-methoxy-1H-benzimidazol-1-yl]acetic acid from the Step A Example 10, 5.7 mg HOBt, and 10.8 mg EDC was added 0.5 mL dry DMF, followed by 6.5 μL di-n-propylamine and 18.2 μL DIEA. This solution was heated at 40° C. over night. It was purified directly on RP-HPLC using 60˜100% MeCN gradient. The fractions containing pure product were pooled and lyophilized to give the title compound. LC-MS: 4.01 minute (M+H=374.4).

### EXAMPLE 14

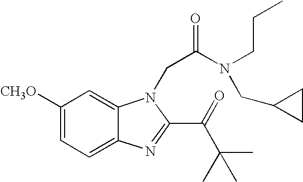

### N-(Cyclopropylmethyl)-2-[2-(2,2-dimethylpropanoyl)-6-methoxy-1H-benzimidazol-1-yl]-N-propylacetamide

To a mixture of 7.3 mg [2-(2,2-dimethylpropanoyl)-6-methoxy-1H-benzimidazol-1-yl]acetic acid from the Step A Example 10, 5.1 mg HOBt, and 9.6 mg EDC was added 0.5 mL dry DMF, followed by 7.4 μL N-propylcyclopropane-methylamine and 16.2 μL DIEA. This solution was heated at 40° C. over night. It was purified directly on RP-HPLC using 60˜100% MeCN gradient. The fractions containing pure product were pooled and lyophilized to give the title compound. LC-MS: 4.05 minute (M+H=386.4).

### EXAMPLE 15

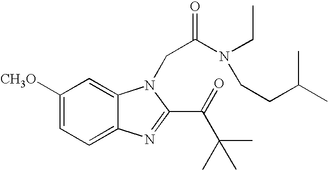

### 2-[2-(2,2-Dimethylpropanoyl)-6-methoxy-1H-benzimidazol-1-yl]-N-ethyl-N-(3-methylbutyl)acetamide

To a mixture of 7.2 mg [2-2,2-dimethylpropanoyl)-6-methoxy-1H-benzimidazol-1-yl]acetic acid from the Step A Example 10, 5.0 mg HOBt, and 9.5 mg EDC was added 0.5 mL dry DMF, followed by 5.7 μL N-ethyl-1-amylamine and 16 μL DIEA. This solution was heated at 40° C. over night. It was purified directly on RP-HPLC using 65˜100% MeCN gradient. The fractions containing pure product were pooled and lyophilized to give the title compound. LC-MS: 4.15 minute (M+H=388.4).

### EXAMPLE 16

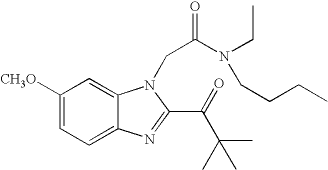

### N-Butyl-2-[2-(2,2-dimethylpropanoyl)-6-methoxy-1H-benzimidazol-1-yl]-N-ethylacetamide

To a mixture of 7.2 mg [2-(2,2-dimethylpropanoyl)-6-methoxy-1H-benzimidazol-1-yl]acetic acid from the Step A Example 10, 5.0 mg HOBt, and 9.5 mg EDC was added 0.5 mL dry DMF, followed by 5.2 μL N-ethylbutylamine and 16.0 μL DIEA. This solution was heated at 40° C. over night. It was purified directly on RP-HPLC using 60˜100% MeCN gradient. The fractions containing pure product were pooled and lyophilized to give the title compound. LC-MS: 4.02 minute (M+H=374.4).

### EXAMPLE 17

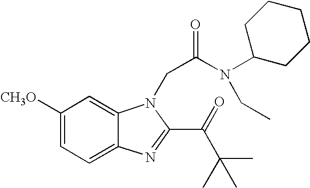

### N-Cyclohexyl-2-[2-(2,2-dimethylpropanoyl)-6-methoxy-1H-benzimidazol-1-yl]-N-ethylacetamide

To a mixture of 7.6 mg [2-(2,2-dimethylpropanoyl)-6-methoxy-1H-benzimidazol-1-yl]acetic acid from the Step A Example 10, 5.3 mg HOBt, and 10.0 mg EDC was added 0.5 mL dry DMF, followed by 6.3 μL N-ethylcyclohexylamine and 16.9 μL DIEA. This solution was heated at 40° C. over night. It was purified directly on RP-HPLC using 65˜100% MeCN gradient. The fractions containing pure product were pooled and lyophilized to give the title compound. LC-MS: 4.20 minute (M+H=400.4).

### EXAMPLE 18

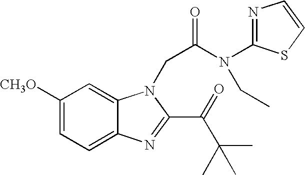

### 2-[2-(2,2-Dimethylpropanoyl)-6-methoxy-1H-benzimidazol-1-yl]-N-ethyl-N-1,3-thiazol-2-ylacetamide

To a mixture of 7.1 mg [2-2,2-dimethylpropanoyl)-6-methoxy-1H-benzimidazol-1-yl]acetic acid from the Step A Example 10, 4.7 mg N-ethyl-1,3-thiazol-2-amine from the Step A Example 9, 5.0 mg HOBt, and 9.4 mg EDC was added 0.5 mL dry DMF, followed by 15.8 μL DIEA. This solution was heated at 40° C. over night. It was purified directly on RP-HPLC using 55˜100% MeCN gradient. The fractions containing pure product were pooled and lyophilized to give the title compound. LC-MS: 3.94 minute (M+H=401.4).

### EXAMPLE 19

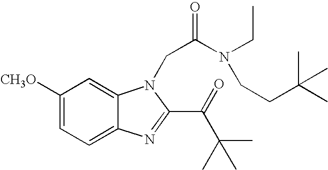

### N-(3,3-Dimethylbutyl)-2-[2-(2,2-dimethylpropanoyl)-6-methoxy-1H-benzimidazol-1-yl]-N-ethylacetamide Step A: N-Ethyl-3,3-dimethylbutan-1-amine hydrochloride

The title compound was prepared from commercially available ethylamine and 3,3-dimethylbutyraldehye using sodium triacetoxyborohydride (Abdel-Magid, et al. J. Org. Chem. 1996, 61, 3849). ¹H NMR (CD₃OD, 500 MHz) δ 3.07 (q, 7.1 Hz, 2H), 2.97˜3.02 (m, 2H), 1.57˜1.62 (m, 2H), 1.32 (t, 7.2 Hz, 3H), 0.98 (s, 9H).

### Step B: N-(3,3-Dimethylbutyl)-2-[2-(2,2-dimethylpropanoyl)-6-methoxy-1H-benzimidazol-1-yl]-N-ethylacetamide

To a mixture of 16.4 mg [2-2,2-dimethylpropanoyl)-6-methoxy-1H-benzimidazol-1-yl]acetic acid from the Step A Example 10, 13.9 mg N-ethyl-3,3-dimethylbutan-1-amine hydrochloride from the Step A above, 11.4 mg HOBt, and 21.5 mg EDC was added 0.5 mL dry DMF, followed by 49 μL DIEA. This solution was heated at 52° C. for 2.5 hours. It was purified directly on RP-HPLC using 65˜100% MeCN gradient. The fractions containing pure product were pooled and lyophilized to give the title compound. In ¹H NMR, it showed two sets of signals in about 1:1 ratio due to slow rotation of the C—N bond in the amide. ¹H NMR (CDCl₃) δ 7.77 & 7.76 (d, J=8.9 Hz, 1H), 6.99 & 6.98 (dd, J=2.8 & 8.9 Hz, 1H), 6.69 (d, J=2.2 Hz, 1H), 5.30 & 5.28 (s, 2H), 3.90 & 3.89 (s, 3H), 3.35˜3.52 (m, 4H), 1.68˜1.72 & 1.45˜1.49 (m, 2H), 1.54 (s, 9H), 1.39 & 1.16 (t, J=7.1 Hz, 3H), 1.05 & 0.93 (s, 9H). LC-MS: 4.27 minute (M+H=402.0).

### EXAMPLE 20

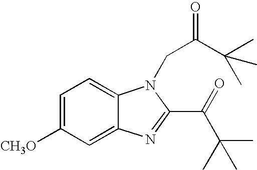

### 1-[2-(2,2-Dimethylpropanoyl)-5-methoxy-1H-benzimidazol-1-yl]-3,3-dimethylbutan-2-one

A mixture of 26 mg 1-(6-methoxy-1H-benzimidazol-2-yl)-2,2-dimethylpropan-1-one from Example 1 Step C and 43 mg cesium carbonate in 1 mL dry DMF was treated with 25.1 mg 1-bromo-3,3-dimethylbutan-2-one at 40° C. over night. After workup, the title compound was isolated from silica gel chromatography (7:1 hexanes and ethyl acetate) as the fast-eluting isomer. The identity of this isomer was confirmed by NOE difference spectrum. ¹H NMR (CDCl₃) δ 7.345 (d, J=2.0 Hz, 1H), 7.09 (d, J=8.9 Hz, 1H), 7.05 (dd, J=2.3 & 8.9 Hz, 1H), 5.54 (s, 2H), 3.90 (s, 3H), 1.54 (s, 9H), 1.35 (s, 9H). LC-MS: 3.92 minute (M+H=331.4).

### EXAMPLE 21

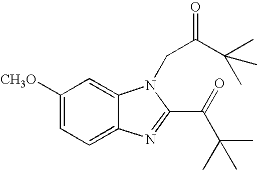

### 1-[2-2,2-Dimethylpropanoyl)-6-methoxy-1H-benzimidazol-1-yl]-3,3-dimethylbutan-2-one

The title compound was separated from the mixture producing Example 20 as the slow-eluting isomer. The identity of this isomer was confirmed by NOE difference spectrum. ¹H NMR (CDCl₃) δ 7.79 (d, J=9.0 Hz, 1H), 7.00 (dd, J=2.3 & 9.0 Hz, 1H), 6.56 (d, J=2.3 Hz, 1H), 5.52 (s, 2H), 3.88 (s, 3H), 1.53 (s, 9H), 1.36 (s, 9H). LC-MS: 3.99 minute (M+H=331.4).

### EXAMPLE 22

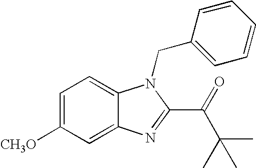

### 1-(1-Benzyl-5-methoxy-1H-benzimidazol-2-yl)-2,2-dimethylpropan-1-one

The mixture from Example 1 Step B can be separated on RP-HPLC to give the title compound as fast-eluting isomer. Its identity was assigned based on NOE difference spectrum. ¹H NMR (CDCl₃, 500 MHz) δ 7.40 (d, J=2.3 Hz, 1H), 7.28˜7.33 (m, 4H), 7.08˜7.11 (m, 3H), 5.69 (s, 2H), 3.91 (s, 3H), 1.43 (s, 9H).

### EXAMPLE 23

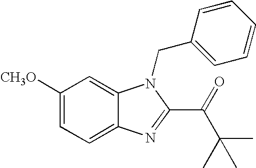

### 1-(1-Benzyl-6-methoxy-1H-benzimidazol-2-yl)-2,2-dimethylpropan-1-one

The mixture from Example 1 Step B can be separated on RP-HPLC to give the title compound as slow-eluting isomer. Its identity was assigned based on NOE difference spectrum. ¹H NMR (CDCl₃, 500 MHz) δ 7.85 (d, J=9.0 Hz, 1H), 7.28˜7.34 (m, 3H), 7.08˜7.11 (m, 3H), 6.81 (d, J=2.3 Hz, 1H), 5.69 (s, 2H), 3.86 (s, 3H), 1.39 (s, 9H).

### EXAMPLE 24

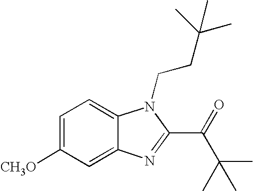

### 1-[1-(3,3-Dimethylbutyl)-5-methoxy-1H-benzimidazol-2-yl]-2,2-dimethylpropan-1-one

### Step A. 1-(3,3-Dimethylbutyl)-5-methoxy-1H-benzimidazole and 1-(3,3-dimethylbutyl)-6-methoxy-1H-benzimidazole

To a mixture of 4.23 g 5-methoxy-1H-bezimidazole and 11.7 g cesium carbonate in 30 mL DMF was added 5.9 g 1-bromo-3,3-dimethylbutane. After stirring the mixture at room temperature over night, it was quenched by addition of saturated ammonium chloride solution. It was diluted with water and extracted with ethyl acetate. The ethyl acetate solution was washed with saturated brine, dried over anhydrous Na₂SO₄ and evaporated under reduced pressure to give the title compound. ¹H NMR (CDCl₃, 500 MHz) of 1-(3,3-dimethylbutyl)-5-methoxy-1H-benzimidazole δ 7.90 (s, 1H), 7.30 (d, J=2.3 Hz, 1H), 7.28 (d, J=8.9 Hz, H), 6.98 (dd, J=2.3 & 8.9 Hz, 1H), 4.15˜4.18 (m, 2H), 3.89 (s, 3H), 1.79˜1.83 (m, 2H), 1.06 (s, 9H). ¹H NMR (CDCl₃, 500 MHz) of 1-(3,3-dimethylbutyl)-6-methoxy-1H-benzimidazole δ 7.83 (s, 1H), 7.70 (d, J=8.9 Hz, 1H), 6.94 (dd, J=2.3 & 8.9 Hz, 1H), 6.835 (d, J=2.3 Hz, 1H), 4.10˜4.15 (m, 2H), 3.90 (s, 3H), 1.78˜1.82 (m, 2H), 1.06 (s, 9H).

### Step B. 1-[1-(3,3-Dimethylbutyl)-5-methoxy-1H-benzimidazol-2-yl]-2,2-dimethylpropan-1-one

To a solution of 0.825 g product from the Step A above in 10 mL anhydrous THF cooled with an acetone-dry ice bath at −78° C. was added 2.2 mL 2.0 M lithium diisopropylamide in heptane/tetrahydrofuran/ethylbenzene. The resulting mixture was stirred for 20 minutes and treated with 0.505 g N,N,2,2-tetramethylpropanamide. After stirring the reaction mixture in the cooling bath for 15 minutes, the cooling bath was removed and the reaction mixture was allowed to warm to room temperature. It was quenched by addition of saturated ammonium chloride solution, diluted with water and extracted with ethyl acetate. The ethyl acetate solution was washed with saturated brine, dried over anhydrous Na₂SO₄ and evaporated under reduced pressure. The title compound was isolated from the crude mixture as the fast-eluting isomer by RP-HPLC. ¹H NMR (CDCl₃, 500 MHz) δ 7.44 (br s, 1H), 7.33 (d, J=9.2 Hz, 1H), 7.13 (dd, J=2.2 & 9.0 Hz, 1H), 4.46˜4.49 (m, 2H), 3.92 (s, 3H), 1.70˜1.74 (m, 2H), 1.56 (s, 9H), 1.09 (s, 9H). LC-MS: 4.44 minute (M+H=317.3). Its identity was assigned based on NOE difference spectrum.

### EXAMPLE 25

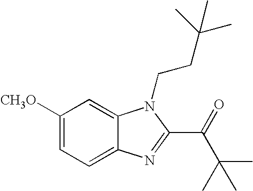

### 1-[1-(3,3-Dimethylbutyl)-6-methoxy-1H-benzimidazol-2-yl]-2,2-dimethylpropan-1-one

The title compound was isolated from the crude mixture from Step B Example 24 as the slow-eluting isomer by RP-HPLC. ¹H NMR (CDCl₃, 500 MHz) δ 7.93 (d, J=9.1 Hz, 1H), 7.24 (dd, J=2.1 & 9.2 Hz, 1H), 6.87 (d, J=2.3 Hz, 1H), 4.40˜4.44 (m, 2H), 3.96 (s, 3H), 1.77˜1.80 (m, 2H), 1.50 (s, 9H), 1.11 (s, 914). LC-MS: 4.53 minute (M+H=317.2). Its identity was assigned based on NOE difference spectrum.

Scheme 3

Compounds of formula I, can be prepared as shown in Scheme 3 by reacting diaminoanisole and carboxylic acid in 4N HCl (Ramaiah, K.; Grossert, J. S.; Hooper, D. L.; Dubey, P. K.; Ramanatham, J.; J Indian Chem Soc 1999, 76 (3), 140-144.), or polyphosphoric acid (PPA) at 130° C. (Walker, A. M.; Craig, J. C.; Fu, C. C.; Ekwuribe, N. N.; Synthesis 1981, 303.). Compound I was alkylated with bromopinacolone and separated the regio isomers to give compounds IIa and IIb. Compounds IIIa and IIIb were obtained in similar manner. Alkylation of compound I with tert-Butyl chloroacetate followed by separation to give two regio isomers. Converting tert-butyl ester to corresponding carboxylic acid, which was coupled with dialkylamine to give compounds IIIa and IIIb.

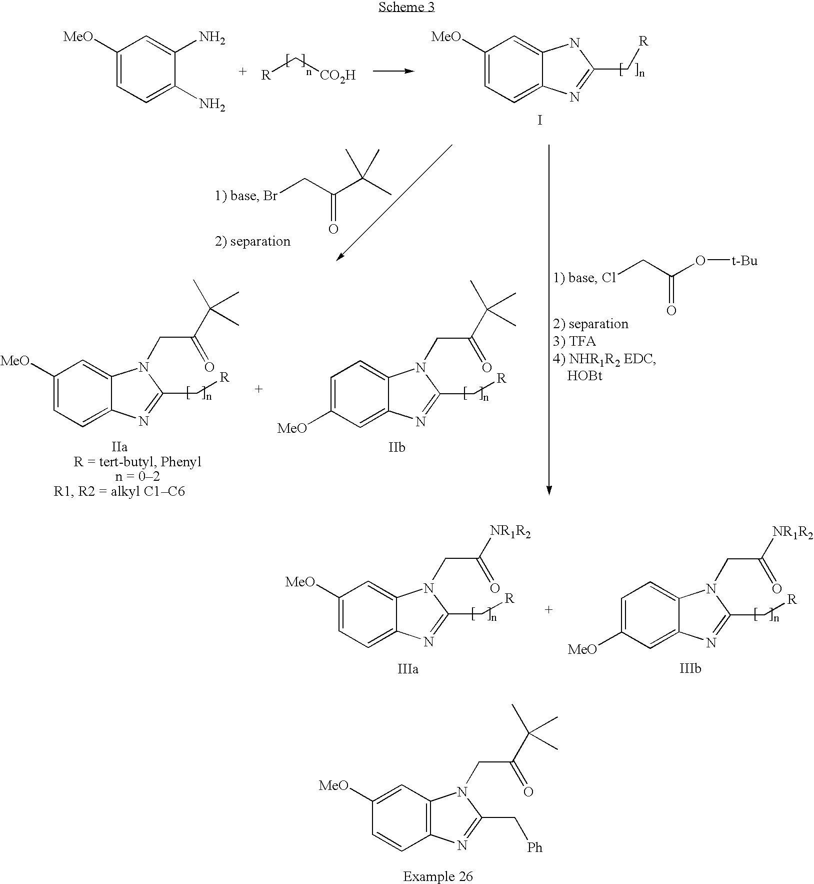

### 1-(2-benzyl-6-methoxy-1H-benzimidazol-1-yl)-3,3-dimethylbutan-2-one

Step A:

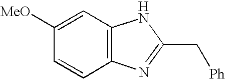

To a solution of 4-methoxy-1,2-phenylenediamine dihydrochloride (2.11 g) and phenylacetic acid (2.04 g) in 4N HCl was refluxed for a couple of hours. After the reaction completed, the mixture was made basic with 1N NaOH and extracted with ethyl acetate. The organic layer was washed with brine and dried over magnesium sulfate. The solution was concentrated and the residue was purified by silica gel (hexanes/ethyl acetate=1/1) to give the desired product (1.2 g). LCMS: (M+H)=239.1.
Step B.

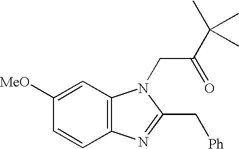

To a solution of the intermediate from Step A (79 mg) in dry DMF was added potassium carbonate (138 mg) and bromopinacolone (0.11 ml). The mixture was heating at 60° C. over night. The mixture was diluted with water and extracted with ethyl acetate. The ethyl acetate solution was washed with water (3×), brine, dried over magnesium sulfate and evaporated under reduced pressure. The residue was purified by silica gel (hexanes/ethyl acetate=1.5/1 to 1/1.5) to give 2 regio isomer products. The less polar isomer was identified to be the title compound.

¹H NMR (CDCl₃, 500 MHz): 7.68 (d, 1H), 7.29 (m, 3H), 7.19 (d, 2H), 6.90 (dd, 1H), 6.46 (d, 1H), 4.82 (s, 2H), 4.23 (s, 2H), 3.84 (s, 3H), 1.21 (s, 9H). LCMS: (M+H)=337.3.

### EXAMPLE 27

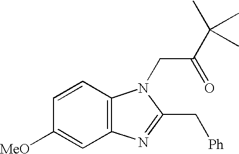

### 1-(2-benzyl-5-methoxy-1H-benzimidazol-1-yl)-3,3-dimethylbutan-2-one

The more polar isomer from Example 26, Step B was isolated and identified to be the title compound. ¹H NMR (CDCl₃, 500 MHz): 7.31 (m, 4H), 7.19 (d, 2H), 6.88 (dd, 2H), 4.85 (s, 2H), 4.23 (s, 2H), 3.88 (s, 3H), 1.20 (s, 9H). LCMS: (M+H)=337.3.

Compounds in example 28-31 were prepared according to the procedure, which was described in examples 26 and 27.

### EXAMPLE 28

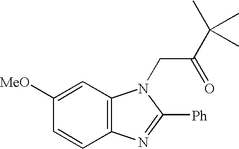

### 1-(6-methoxy-2-phenyl-1H-benzimidazol-1-yl)-3,3-dimethylbutan-2-one

¹H NMR (CDCl₃, 500 M&): 7.74 (d, 1H), 7.57 (m, 2H), 7.49 (m, 3H), 6.96 (dd, 1H), 6.56 (d, 1H), 5.08 (s, 2H), 3.88 (s, 3H), 1.28 (s, 9H). LCMS: (M+H)=323.3.

### EXAMPLE 29

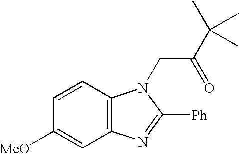

### 1-(5-methoxy-2-phenyl-1H-benzimidazol-1-yl)-3,3-dimethylbutan-2-one

¹H NMR (CDCl₃, 500 MHz): 7.58 (m, 2H), 7.50 (m, 3H), 7.35 (d, 1H), 6.96 (m, 2H), 5.10 (s, 2H), 3.90 (s, 3H), 1.27 (s, 9H). LCMS: (M+H)=323.3.

### EXAMPLE 30

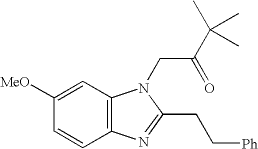

### 1-[6-methoxy-2-(2-phenylethyl)-1H-benzimidazol-1-yl]-3,3-dimethylbutan-2-one

¹H NMR (CDCl₃, 500 MHz): 7.66 (d, 1H), 7.26 (m, 3H), 7.21 (d, 2H), 6.90 (dd, 1H), 6.48 (d, 1H), 4.72 (s, 2H), 3.85 (s, 3H), 3.23 (t, 2H), 2.96 (t, 2H), 1.30 (s, 9H). LCMS: (M+H)=351.3.

### EXAMPLE 31

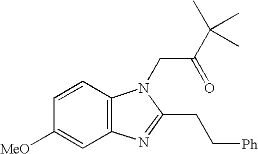

### 1-[5-methoxy-2-(2-phenylethyl)-1H-benzimidazol-1-yl]-3,3-dimethylbutan-2-one

¹H NMR (CDCl₃, 500 MHz): 7.30 (m, 4H), 7.21 (d, 2H), 6.88 (m, 2H), 4.73 (s, 2H), 3.88 (s, 3H), 3.24 (t, 2H), 2.96 (t, 2H), 1.29 (s, 9H). LCMS: (M+H)=351.3.

### EXAMPLE 32

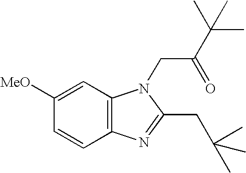

### 1-[2-(2,2-dimethylpropyl)-6-methoxy-1H-benzimidazol-1-yl]-3,3-dimethylbutan-2-one

Step A

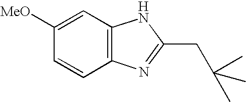

A stirred solution of 4-methoxy-1,2-phenylenediamine dihydrochloride (2.11 g) and tert-butylacetic acid (1.9 ml) in PPA under nitrogen atmosphere was heated at 130° C. for 16 hours. The mixture was cooled to room temperature, poured into iced-water, and the resulting solution was treated with solid sodium carbonate to pH8. The solution was extracted with ethyl acetate. The organic layer was washed with brine and dried over magnesium sulfate. The solution was concentrated and the residue was purified by silica gel.
Step B

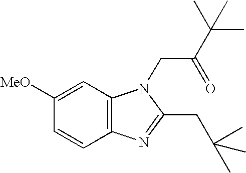

To a solution of the intermediate from Step A (218 mg) in dry DMF was added cesium carbonate (975 mg) and chloropinacolone (0.16 ml). The mixture was stirred at room temperature for a couple of hours. The mixture was diluted with water and extracted with ethyl acetate. The ethyl acetate solution was washed with water (3×), brine, dried over magnesium sulfate and evaporated under reduced pressure. The residue was purified by silica gel (hexanes/ethyl acetate=1/1) to give 2 regio isomer products. The less polar isomer was identified to be the title compound. ¹H NMR (CDCl₃, 500 MHz): 7.66 (d, 1H), 6.88 (dd, 1H), 6.48 (d, 1H), 5.06 (s, 2H), 3.84 (s, 3H), 2.62 (s, 2H), 1.36 (s, 9H), 1.07 (s, 9H).

### EXAMPLE 33

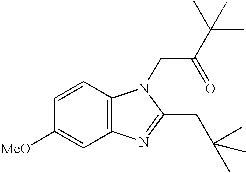

### 1-[2-2,2-dimethylpropyl)-5-methoxy-1H-benzimidazol-1-yl]-3,3-dimethylbutan-2-one

The more polar isomer from Example 32, Step B was isolated and identified to be the title compound. ¹H NMR (CDCl₃, 500 MHz): 7.30 (d, 1H), 6.87 (m, 2H), 5.06 (s, 2H), 3.86 (s, 3H), 2.64 (s, 2H), 1.35 (s, 9H), 1.08 (s, 9H).

### EXAMPLE 34

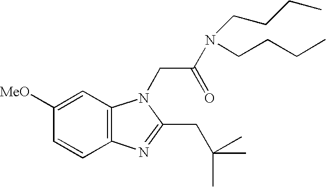

### N,N-dibutyl-2-[2-(2,2-dimethylpropyl)-6-methoxy-1H-benzimidazol-1-yl]acetamide

Step A

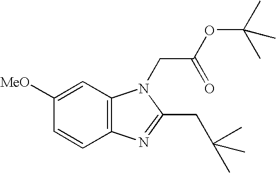

To a solution of the intermediate from Example 32, Step A (218 mg) in dry DMF was added cesium carbonate (975 mg) and tert-butyl chloroacetate (0.17 ml). The mixture was stirred at room temperature for a couple of hours. The mixture was diluted with water and extracted with ethyl acetate. The ethyl acetate solution was washed with water (3×), brine, dried over magnesium sulfate and evaporated under reduced pressure. The residue was purified by silica gel (hexanes/ethyl acetate=2/1 to 1/1) to give 2 regio isomer products. The less polar isomer was identified to be the title compound.
Step B

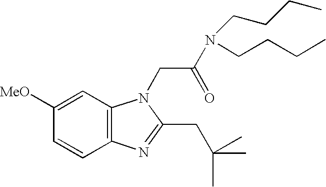

The less polar intermediate from Step A in methylene chloride was added TFA and anisole (0.2 ml) and stirred at room temperature. After reaction completed, the mixture was concentrated to dry. The residue in DMF was added EDC, HOBt, dibutylamine and triethylamine, and, heated to 50° C. for a couple of hours. The mixture was diluted with water and extracted with ethyl acetate. The ethyl acetate solution was washed with water (3×), brine, dried over magnesium sulfate and evaporated under reduced pressure. The residue was purified by silica gel (hexanes/ethyl acetate=1/1 and then hexanes/TBF=2/1) to give the title compound. ¹H NMR (CDCl₃, 500 MHz): 7.66 (d, 1H), 6.89 (dd, 1H), 6.66 (d, 1H), 4.89 (s, 2H), 3.86 (s, 3H), 3.36 (m, 4H), 2.75 (s, 2H), 1.64 (m, 2H), 1.55 (m, 2H), 1.43 (m, 2H), 1.32 (m, 2H), 1.09 (s, 9H), 1.03 (t, 3H), 0.93 (t, 3H).

### EXAMPLE 35

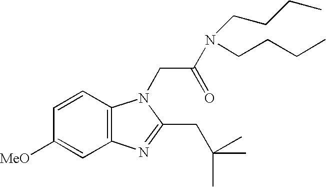

### N,N-dibutyl-2-[2-(2,2-dimethylpropyl)-5-methoxy-1H-benzimidazol-1-yl]acetamide

The more polar intermediate from Example 34, Step A in methylene chloride was added TFA and anisole (0.2 ml) and stirred at room temperature. After reaction completed, the mixture was concentrated to dry. The residue in DMF was added EDC, HOBt, dibutylamine and triethylamine, and heated to 50° C. for a couple of hours. The mixture was diluted with water and extracted with ethyl acetate. The ethyl acetate solution was washed with water (3×), brine, dried over magnesium sulfate and evaporated under reduced pressure. The residue was purified by silica gel (hexanes/ethyl acetate=1/2) to give the title compound. ¹H NMR (CDCl₃, 500 MHz): 7.28 (d, 1H), 7.04 (d, 1H), 6.88 (dd, 1H), 4.89 (s, 2H), 3.86 (s, 3H), 3.35 (m, 4H), 2.74 (s, 2H), 1.64 (m, 2H), 1.54 (m, 2H), 1.41 (m, 2H), 1.31 (m, 2H), 1.10 (s, 9H), 1.03 (t, 3H), 0.93 (t, 3H).

### EXAMPLE 36

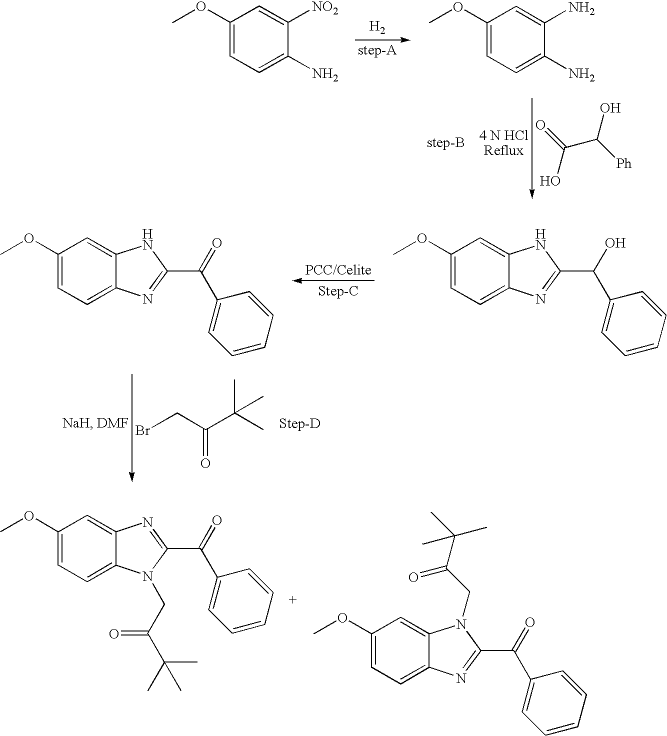 Step-A
20 g of nitro-aniline derivative was dissolved in a mixture of THF and methanol (1/1 v/v) (100 mL). After addition of 10 mol % of Pd—C, the reaction mixture was hydrogenated under pressure in a Parr shaker until the required amount of hydrogen was consumed. TLC analysis indicated completion. The reaction mixture was filtered and evaporated. Crude (14.9 g) used in the next step.
Step-B
The di-amine from above was refluxed with 1.3 equiv. of 2-hydroxy phenyl acetic acid in 50 mL of 4 N HCl for 1 h. The resulting solid precipitate was filtered out after cooling of reaction mixture to room temperature. The desired product (21 g) was dried thoroughly and subjected to oxidation
Step-C
21 g of hydroxy benz-imidazole was dissolved in 200 mL of dichloromethane followed by addition of Celite (2 g/mmole of PCC) and portion-wise addition of PCC (1.5 equiv.). The reaction was complete in 0.5 h. The reaction mixture was filtered and purified over a short plug of silica gel to yield ketone derivative (18 g).
Step-D
To a solution of benzoimidazole (51 mg; 0.2 mmole) in DMF was added NaH (1.68 equiv.). After stirring at rt for 20 min., bromopinacolone (1.1 equiv.) was added. The reaction mixture was stirred at rt for 30 minutes. The reaction was quenched by adding water. Work up with water/EtOAC. The solvent was removed, the residue was purified by reverse phase HPLC to give two regioisomers. MW=350

### EXAMPLES 37-42

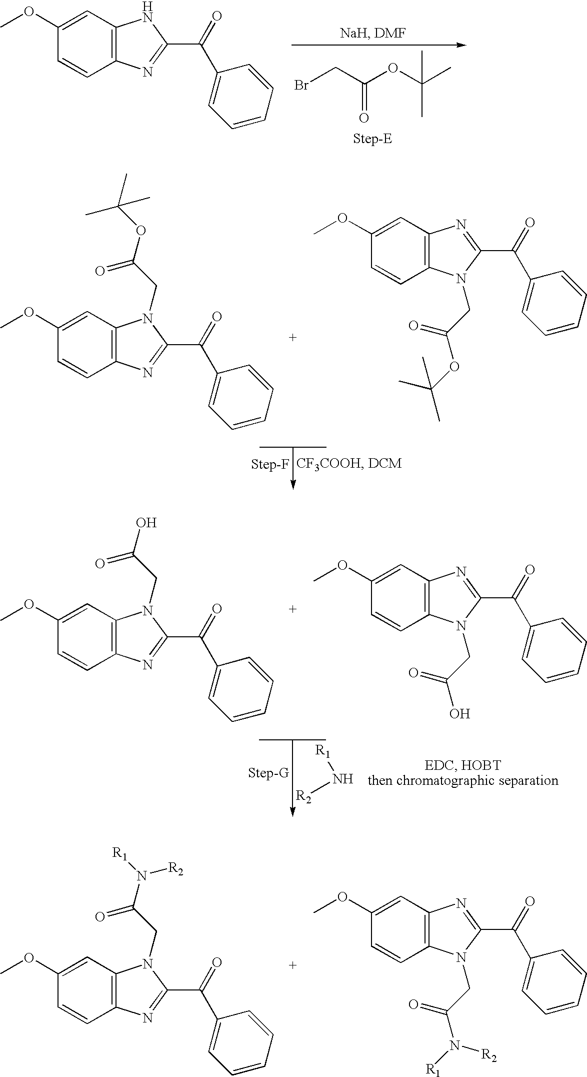 Step E 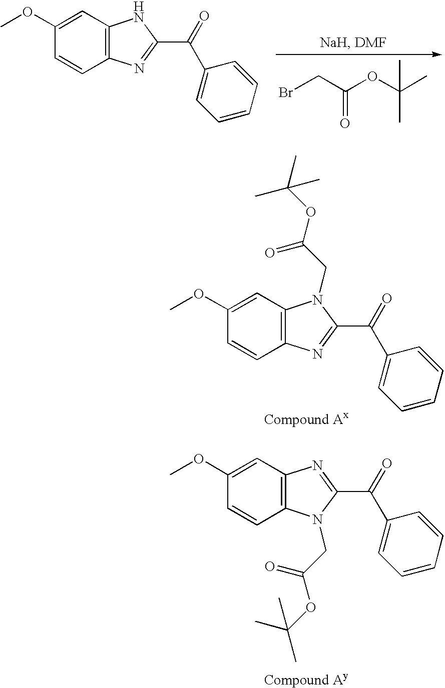
Procedure:

To a solution of benzoimidazole (422 mg/1.67 mmol/1 Eq.) in DMF (10 mL) was added NaH (100×60%=60 mg/2.5 mmol/1.5 Eq). After stirring at rt for 20 min., bromoacetate (d1.32×025 mL=330 mg/1.69 mmol/1.0 Eq.) was added. The reaction mixture was stirred at rt for 30 minutes. The reaction was quenched by adding water. Work up with water/EtOAC. The solvent was removed, the residue was purified by Horizon HPFC (Silica gel column to give 0.352.6 mg yellow solid product.

HNMR show the prod is 2:1 a-b mixture. The mixture was used for next step reaction without further purification.
Step-F 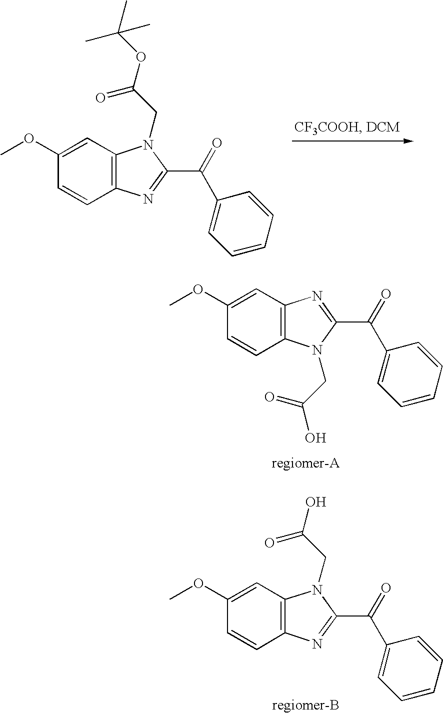
Procedure:

To a solution of Compound A^{x&y} (300 mg) in 1 mL of DCM was added 3 mL TFA. The reaction micture was stirred at rt for 4 hr. The solvent was removed, the residue was used for next step reaction without further purification.

A small portion of the acid was purified by reverse phase HPLC to give two isomers (MW=310).
Step-G 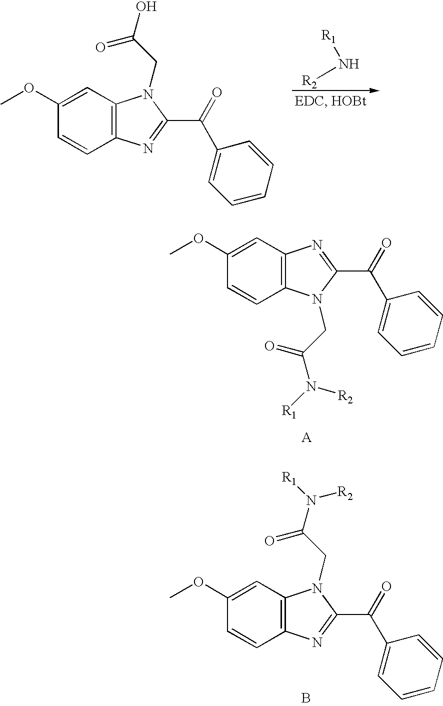
Procedure:

To a solution of acid (310 mg) in 18 mL of CH₃CN was added 235 mg EDC and 111 mg of HOBt. The above solution was divided into 6 reaction tubes (3 mL for each reaction tube) and added different amines to each reaction tube. The reaction mixtures were stirred at rt for 2 hr. and then 1 hr at 75° C. LC-MS showed the reactions were not completed. Added 20 mg of PyBop to each reaction tube and stiring the reaction at 75° C. for addional one hr. The reaction mixtures were purified by reverse phase HPLC to give Amides A and B below.

| 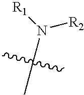 | Example No. |
|---|---|
| 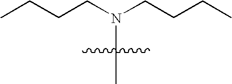 | 37 |
| 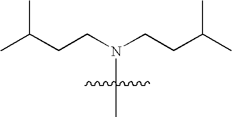 | 38 |
| 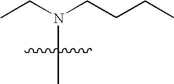 | 39 |
| 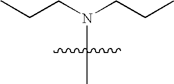 | 40 |
| 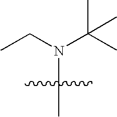 | 41 |
| 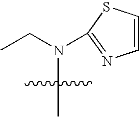 | 42 |

### EXAMPLES 43-45

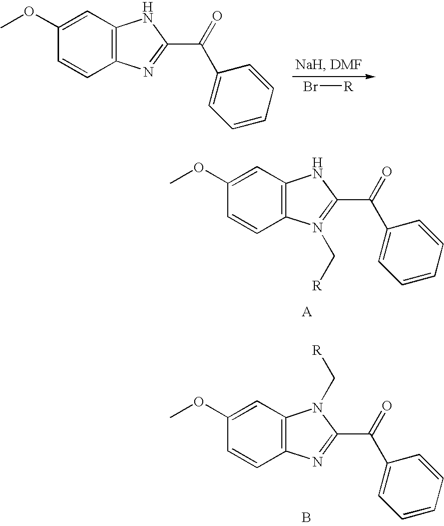

Procedure: To a solution of benzimidazole (50 mg/0.2 mmol/1 Eq) in DMF (1 mL) was added NaH (28 mg×60%=16.8 mg/0.7 mmol/3.5 Eq). After stirring at rt for 20 min., alkylbromide (˜50 mg/˜0.3 mmol/˜1.5 Eq) was added. The reaction mixture was stirred at 70° C. for overnight. The reaction was quenched by adding water and were purified by reverse phase HPLC to give compounds A and B.

| R | Example No. |
|---|---|
| 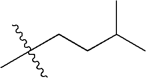 | 43 |
| 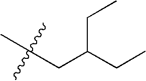 | 44 |
| 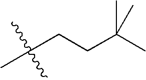 | 45 |

Scheme 4 illustrates the preparation of compounds including a hydroxylbenzyl group.

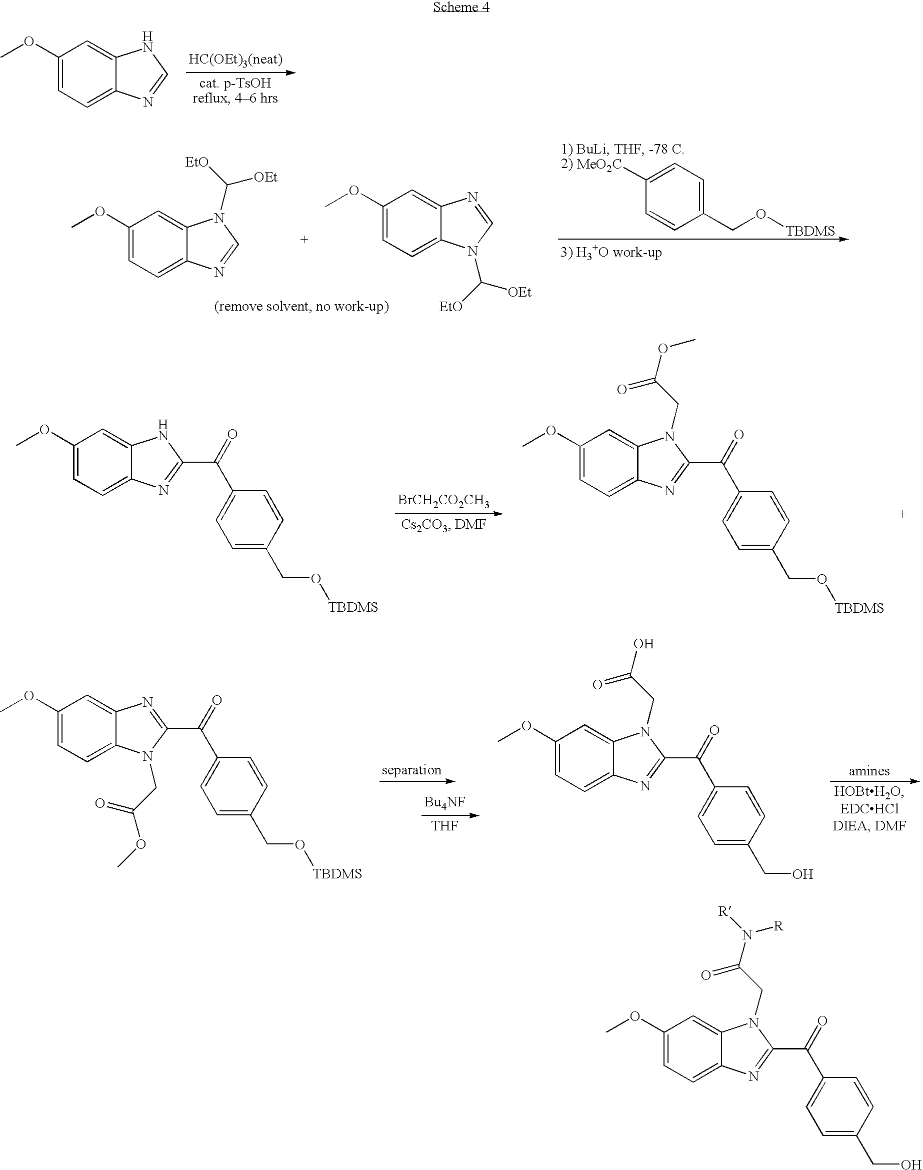

Scheme 5 illustrates the preparation of compounds including both cis- and trans-4-hydroxylcyclohex-1-yl groups.

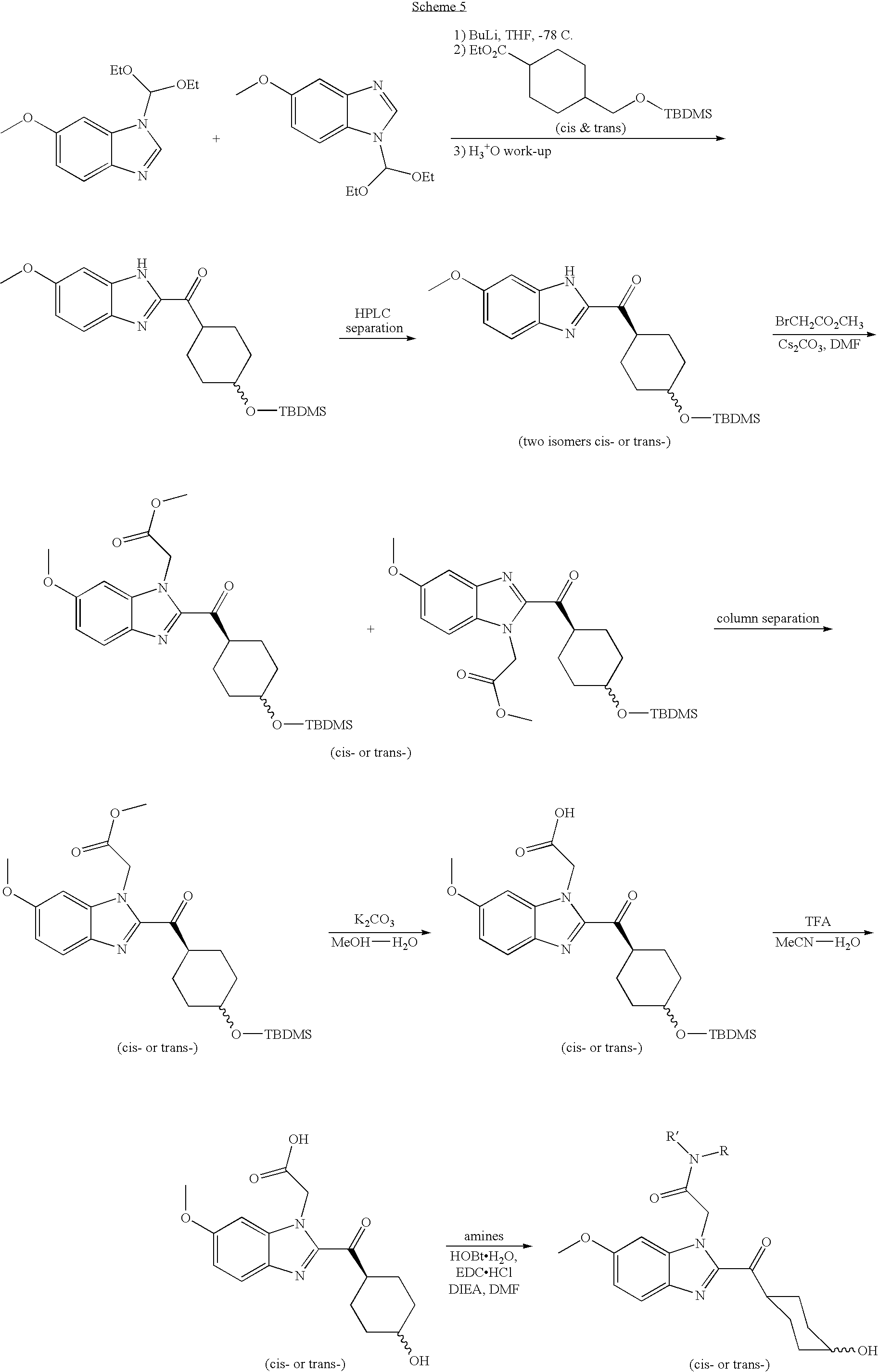

Scheme 6 illustrates the preparation of compounds including both cis- and trans-4-hydroxylmethyl-1-cyclohexyl groups.

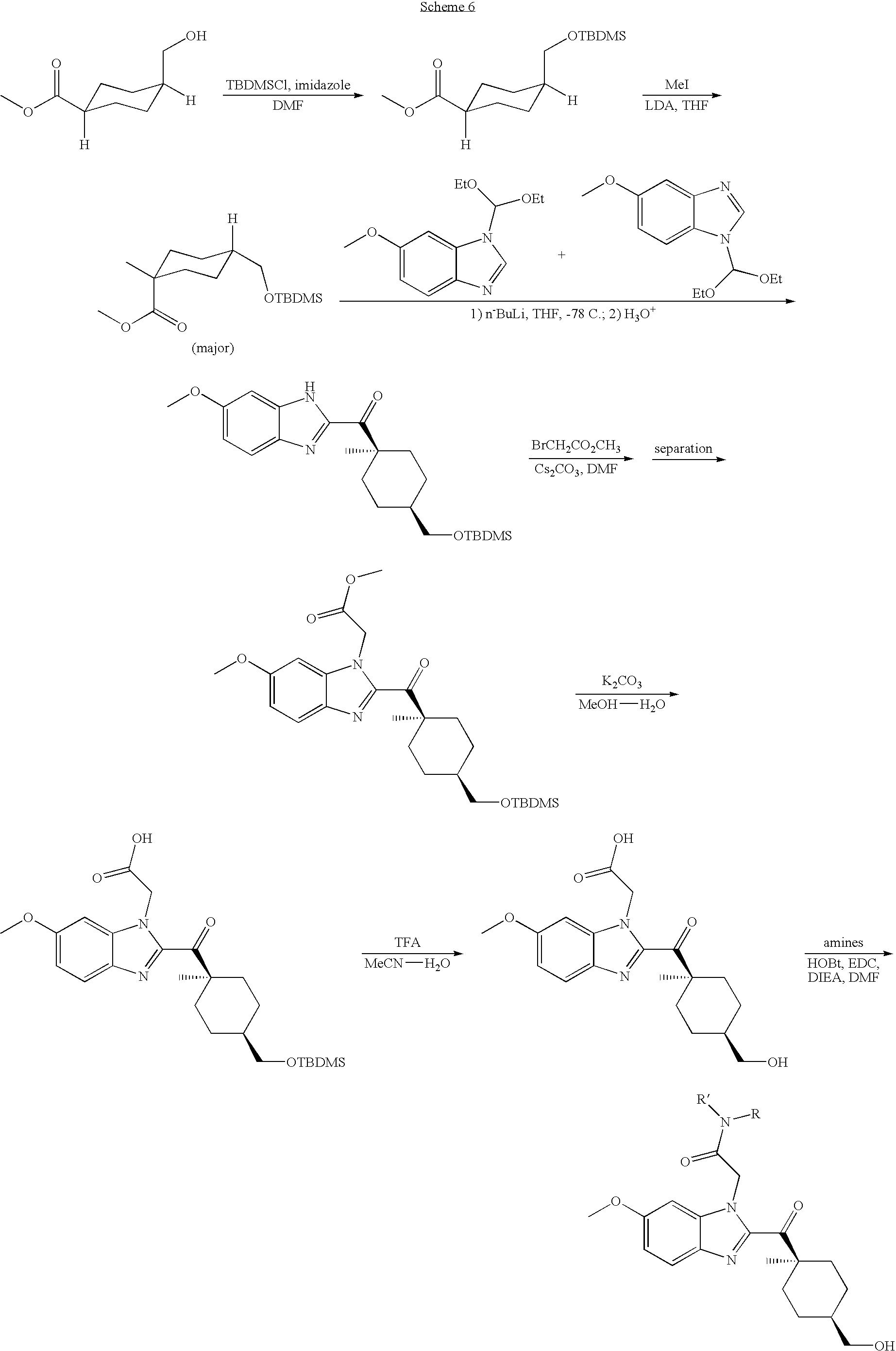

Scheme 7 illustrates the preparation of compounds including 3-hydroxyl-1,1-dimethyl-1-propyl group.

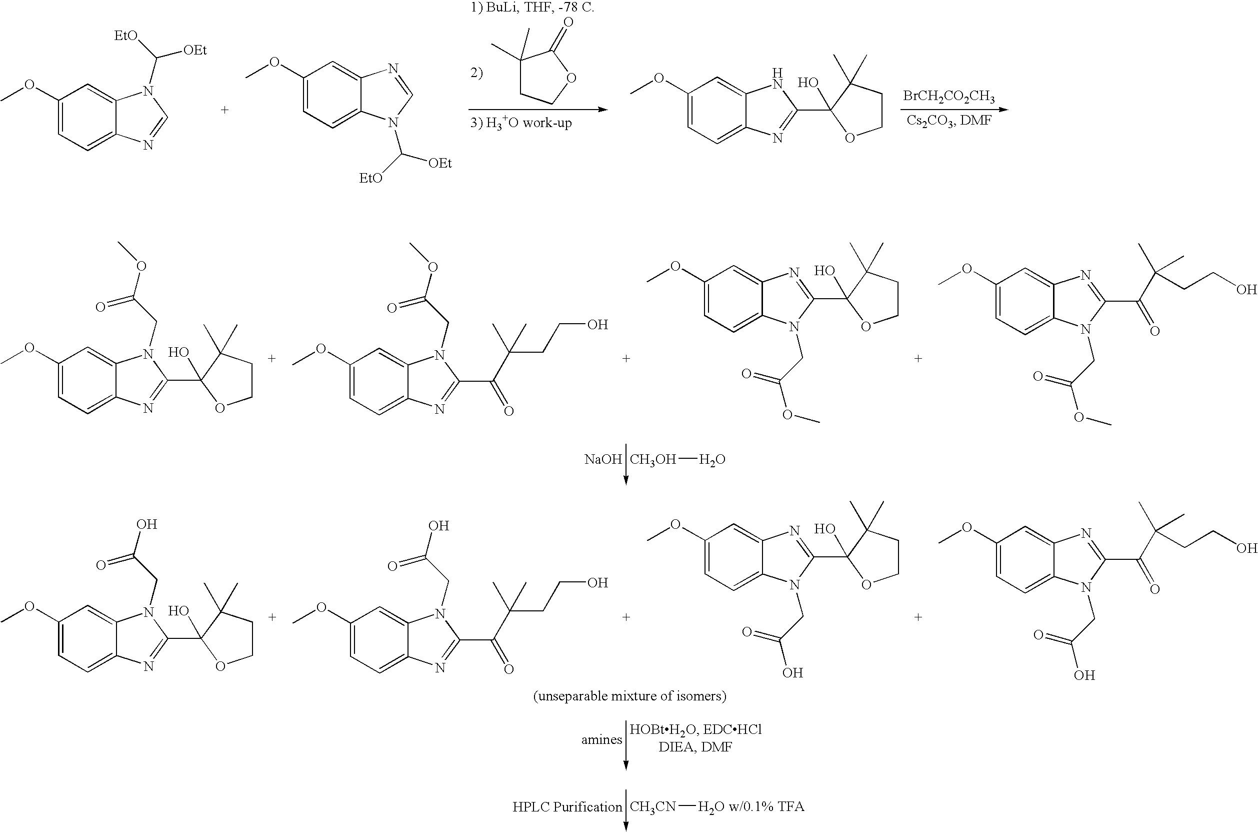

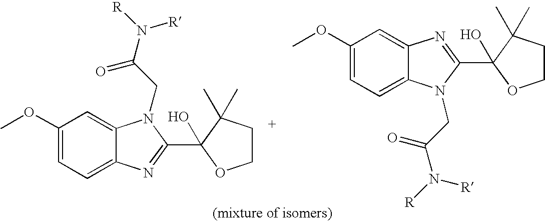

The following Examples in Table 1 were prepared using similar procedures as described for Examples 1˜21 using appropriate substrates.

TABLE 1
Examples 46-63

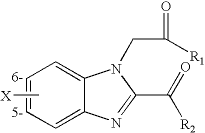

| | | | | LC-MS | |
|---|---|---|---|---|---|
| Example | X | R₁ | R₂ | tᵣ, min. | M + H |
| 46 | 6-MeO | 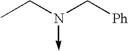 | t-Bu | 3.93 | 408.4 |
| 47 | 6-MeO | 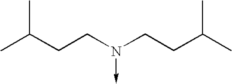 | i-Pr | 4.12 | 416.4 |
| 48 | 6-MeO | 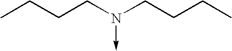 | i-Pr | 3.87 | 388.3 |
| 49 | 6-MeO | 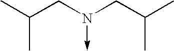 | i-Pr | 3.82 | 318.2 |
| 50 | 6-MeO | 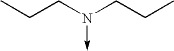 | i-Pr | 3.50 | 290.2 |
| 51 | 6-MeO | 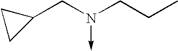 | i-Pr | 3.55 | 302.2 |
| 52 | 6-MeO | 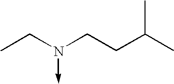 | i-Pr | 3.67 | 304.3 |
| 53 | 6-MeO | 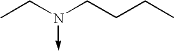 | i-Pr | 3.51 | 290.2 |
| 54 | 6-MeO | 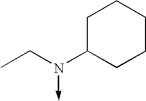 | i-Pr | 3.69 | 316.3 |
| 55 | 6-MeO | 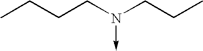 | t-Bu | 4.05 | 388.4 |
| 56 | 6-MeO | 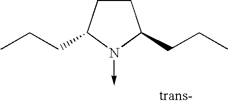 | t-Bu | 4.35 | 428.4 |
| 57 | 6-MeO | 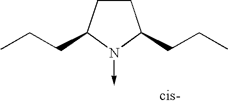 | t-Bu | 4.37 | 428.4 |
| 58 | 5-MeO | t-Bu | i-Pr | 3.51 | 317.3 |
| 59 | 6-MeO | t-Bu | i-Pr | 3.56 | 317.3 |
| 60 | 6-MeO | 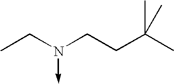 | i-Pr | 3.78 | 388.3 |
| 61 | 6-MeO | 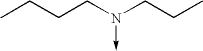 | i-Pr | 3.68 | 374.3 |
| 62 | 6-MeO | 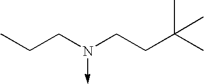 | t-Bu | 4.25 | 416.2 |
| 63 | 6-MeO | 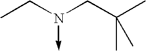 | t-Bu | 4.00 | 388.4 |

The following examples in Table 2 were prepared using the procedure outlined in Scheme 4.

TABLE 2
Examples 64-73

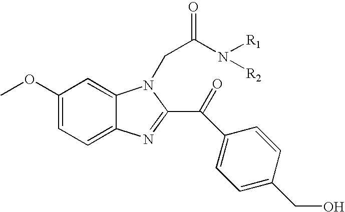

| | | | LC-MS | |
|---|---|---|---|---|
| Example | R₁ | R₂ | tᵣ, min. | M + H |
| 64 | n-Pr | n-Pr | 3.17 | 424.3 |
| 65 | i-Amyl | i-Amyl | 3.76 | 480.3 |
| 66 | n-Bu | n-Bu | 3.49 | 452.3 |
| 67 | i-Bu | i-Bu | 3.46 | 452.3 |
| 68 | n-Bu | n-Pr | 3.34 | 438.3 |
| 69 | n-Pr | cyclopropylmethyl | 3.21 | 436.3 |
| 70 | ethyl | 3,3-dimethylbutyl | 3.46 | 452.3 |
| 71 | ethyl | i-Amyl | 3.33 | 438.3 |
| 72 | ethyl | n-Bu | 3.18 | 424.3 |
| 73 | ethyl | cyclohexyl | 3.34 | 450.3 |

The following examples in Table 3 were prepared using the procedure outlined in Scheme 5.

TABLE 3
Examples 74-86

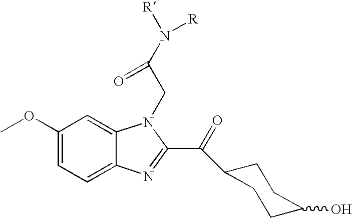

| | | | | LC-MS | |
|---|---|---|---|---|---|
| | | | | tᵣ, | |
| Example | cyclohexane | R | R′ | min. | M + H |
| 74 | trans | i-Amyl | i-Amyl | 3.65 | 472.3 |
| 75 | trans | n-Bu | n-Bn | 3.38 | 444.3 |
| 76 | trans | n-Bu | n-Pr | 3.20 | 430.2 |
| 77 | trans | i-Bu | i-Bu | 3.34 | 444.2 |
| 78 | trans | n-Pr | 3,3-dimethylbutyl | 3.50 | 458.2 |
| 79 | trans | n-Pr | cyclopropylmethyl | 3.10 | 428.2 |
| 80 | trans | ethyl | 3,3-dimethylbutyl | 3.34 | 444.2 |
| 81 | trans | ethyl | 2,2-dimethylpropyl | 3.18 | 430.2 |
| 82 | trans | n-Pr | n-Pr | 3.05 | 416.2 |
| 83 | trans | ethyl | n-Bu | 3.06 | 416.2 |
| 84 | trans | ethyl | cyclohexyl | 3.23 | 442.2 |
| 85 | cis | i-Amyl | i-Amyl | 3.71 | 472.4 |
| 86 | cis | n-Bu | n-Bu | 3.41 | 444.3 |

TABLE 4
Examples 87-103

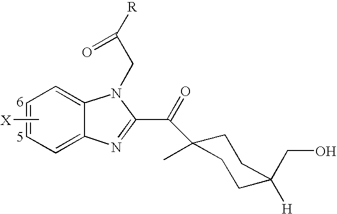

| | | | LC-MS | |
|---|---|---|---|---|
| Exam- | | | tᵣ, | |
| ple | X | R | min. | M + H |
| 87 | 6-MeO | t-Bu | 3.60 | 401 |
| 88 | 6-MeO | 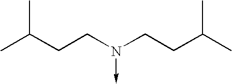 | 4.12 | 500.2 |
| 89 | 6-MeO | 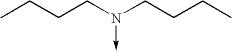 | 3.88 | 472.2 |
| 90 | 6-MeO | 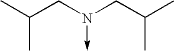 | 3.85 | 472.2 |
| 91 | 6-MeO | 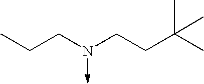 | 3.97 | 486.2 |
| 92 | 6-MeO | 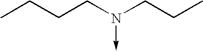 | 3.74 | 458.2 |
| 93 | 6-MeO | 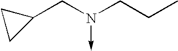 | 3.60 | 456.1 |
| 94 | 6-MeO | 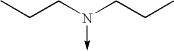 | 3.61 | 444.4 |
| 95 | 6-MeO | 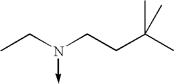 | 3.86 | 472.3 |
| 96 | 6-MeO | 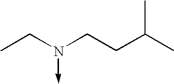 | 3.77 | 458.2 |
| 97 | 6-MeO | 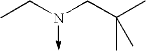 | 3.71 | 458.3 |
| 98 | 6-MeO | 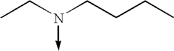 | 3.61 | 444.4 |
| 99 | 6-MeO | 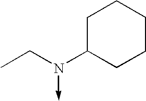 | 3.76 | 470.2 |
| 100 | 6-MeO | 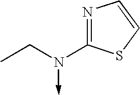 | 3.41 | 471 |
| 101 | 5-MeO | 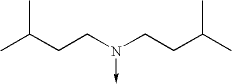 | 4.16 | 500.3 |
| 102 | 5-MeO | 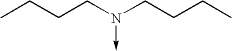 | 3.90 | 472.4 |
| 103 | 5-MeO | 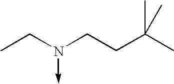 | 3.84 | 472.4 |

Scheme 8 illustrates the preparation of compounds having azabenzoimidazole core structures.

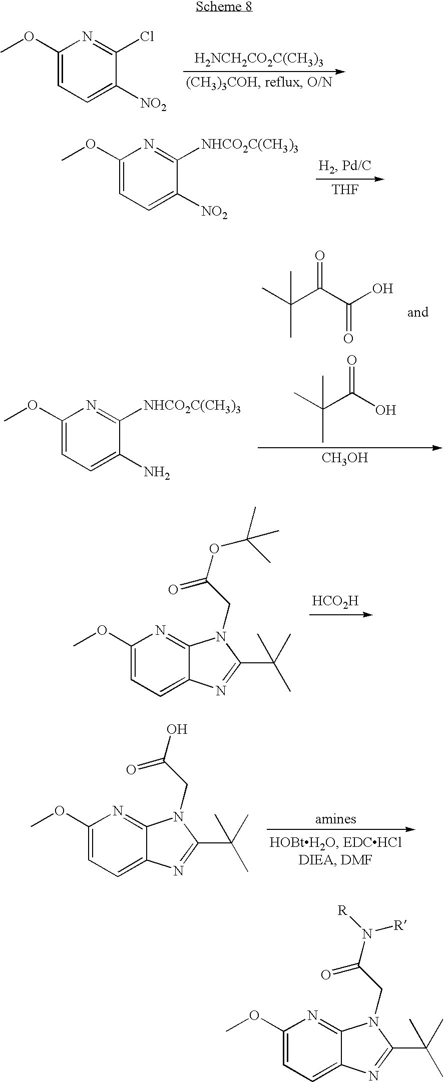

TABLE 5
Examples 104-111

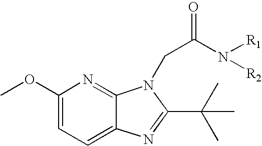

| | | | LC-MS | |
|---|---|---|---|---|
| Example | R₁ | R₂ | tᵣ, min. | M + H |
| 104 | i-Amyl | i-Amyl | 3.28 | 403.1 |
| 105 | n-Bu | n-Bu | 3.02 | 375.1 |
| 106 | i-Bu | i-Bu | 2.98 | 375.1 |
| 107 | n-Pr | 3,3-dimethylbutyl | 3.12 | 389.1 |
| 108 | n-Pr | cyclopropylmethyl | 2.73 | 359.1 |
| 109 | ethyl | 3,3-dimethylbutyl | 2.83 | 361.1 |
| 110 | ethyl | i-Amyl | 2.85 | 361.1 |
| 111 | ethyl | cyclohexyl | 2.87 | 373.1 |

Functional Assays
A. Maxi-K Channel
The activity of the compounds can also be quantified by the following assay.

The identification of inhibitors of the Maxi-K channel is based on the ability of expressed Maxi-K channels to set cellular resting potential after transfection of both alpha and beta1 subunits of the channel in HEK-293 cells and after being incubated with potassium channel blockers that selectively eliminate the endogenous potassium conductances of HEK-293 cells. In the absence of maxi-K channel inhibitors, the transfected HEK-293 cells display a hyperpolarized membrane potential, negative inside, close to E_{K} (−80 mV) which is a consequence of the activity of the maxi-K channel. Blockade of the Maxi-K channel by incubation with maxi-K channel blockers will cause cell depolarization. Changes in membrane potential can be determined with voltage-sensitive fluorescence resonance energy transfer (FRET) dye pairs that use two components, a donor coumarin (CC₂DMPE) and an acceptor oxanol (DiSBAC₂(3)).

Oxanol is a lipophilic anion and distributes across the membrane according to membrane potential. Under normal conditions, when the inside of the cell is negative with respect to the outside, oxanol is accumulated at the outer leaflet of the membrane and excitation of coumarin will cause FRET to occur. Conditions that lead to membrane depolarization will cause the oxanol to redistribute to the inside of the cell, and, as a consequence, to a decrease in FRET. Thus, the ratio change (donor/acceptor) increases after membrane depolarization, which determines if a test compound actively blocks the maxi-K channel.

The HEK-293 cells were obtained from the American Type Culture Collection, 12301 Parklawn Drive, Rockville, Md., 20852 under accession number ATCC CRL-1573. Any restrictions relating to public access to the microorganism shall be irrevocably removed upon patent issuance.

Transfection of the alpha and beta1 subunits of the maxi-K channel in HEK-293 cells was carried out as follows: HEK-293 cells were plated in 100 mm tissue culture treated dishes at a density of 3×10⁶ cells per dish, and a total of five dishes were prepared. Cells were grown in a medium consisting of Dulbecco's Modified Eagle Medium (DMEM) supplemented with 10% Fetal Bovine serum, 1× L-Glutamine, and 1× Penicillin/Streptomycin, at 37° C., 10% CO₂. For transfection with Maxi-K hα(pCIneo) and Maxi-K hβ1(pMESpuro) DNAs, 150 μl FuGENE6™ was added dropwise into 10 ml of serum free/phenol-red free DMEM and allowed to incubate at room temperature for 5 minutes. Then, the FuGENE6™ solution was added dropwise to a DNA solution containing 25 μg of each plasmid DNA, and incubated at room temperature for 30 minutes. After the incubation period, 2 ml of the FuGENE6™/DNA solution was added dropwise to each plate of cells and the cells were allowed to grow two days under the same conditions as described above. At the end of the second day, cells were put under selection media which consisted of DMEM supplemented with both 600 μg/ml G418 and 0.75 μg/ml puromycin. Cells were grown until separate colonies were formed. Five colonies were collected and transferred to a 6 well tissue culture treated dish. A total of 75 colonies were collected. Cells were allowed to grow until a confluent monolayer was obtained. Cells were then tested for the presence of maxi-K channel alpha and beta1 subunits using an assay that monitors binding of ¹²⁵I-iberiotoxin-D19Y/Y36P to the channel. Cells expressing ¹²⁵I-iberiotoxin-D19Y/Y36F binding activity were then evaluated in a functional assay that monitors the capability of maxi-K channels to control the membrane potential of transfected HEK-293 cells using fluorescence resonance energy transfer (FRET) ABS technology with a VIPR instrument. The colony giving the largest signal to noise ratio was subjected to limiting dilution. For this, cells were resuspended at approximately 5 cells/ml, and 200 μl were plated in individual wells in a 96 well tissue culture treated plate, to add ca. one cell per well. A total of two 96 well plates were made. When a confluent monolayer was formed, the cells were transferred to 6 well tissue culture treated plates. A total of 62 wells were transferred. When a confluent monolayer was obtained, cells were tested using the FRET-functional assay. Transfected cells giving the best signal to noise ratio were identified and used in subsequent functional assays.

For Functional Assays:

The transfected cells (2E+06 Cells/mL) are then plated on 96-well poly-D-lysine plates at a density of about 100,000 cells/well and incubated for about 16 to about 24 hours. The medium is aspirated of the cells and the cells washed one time with 100 μl of Dulbecco's phosphate buffered saline (D-PBS). One hundred microliters of about 9 μM coumarin (CC₂DMPE)-0.02% pluronic-127 in D-PBS per well is added and the wells are incubated in the dark for about 30 minutes. The cells are washed two times with 100 μl of Dulbecco's phosphate-buffered saline and 100 μl of about 4.5 μM of oxanol (DiSBAC₂(3)) in (mM) 140 NaCl, 0.1 KCl, 2 CaCl₂, 1 MgCl₂, 20 Hepes-NaOH, pH 7.4, 10 glucose is added. Three micromolar of an inhibitor of endogenous potassium conductance of HEK-293 cells is added. A maxi-K channel blocker is added (about 0.01 micromolar to about 10 micromolar) and the cells are incubated at room temperature in the dark for about 30 minutes.

The plates are loaded into a voltage/ion probe reader (VIPR) instrument, and the fluorescence emission of both CC₂DMPE and DiSBAC₂(3) are recorded for 10 sec. At this point, 100 μl of high-potassium solution (mM): 140 KCl, 2 CaCl₂, 1 MgCl₂, 20 Hepes-KOH, pH 7.4, 10 glucose are added and the fluorescence emission of both dyes recorded for an additional 10 sec. The ratio CC₂DMPE/DiSBAC2(3), before addition of high-potassium solution equals 1. In the absence of maxi-K channel inhibitor, the ratio after addition of high-potassium solution varies between 1.65-2.0. When the Maxi-K channel has been completely inhibited by either a known standard or test compound, this ratio remains at 1. It is possible, therefore, to titrate the activity of a Maxi-K channel inhibitor by monitoring the concentration-dependent change in the fluorescence ratio.

The compounds of this invention were found to cause concentration-dependent inhibition of the fluorescence ratio with IC₅₀'s in the range of about 1 nM to about 20 μM, more preferably from about 10 nM to about 500 nM.

B. Electrophysiological Assays of Compound Effects on High-Conductance Calcium-Activated Potassium Channels

Methods:

Patch clamp recordings of currents flowing through large-conductance calcium-activated potassium (maxi-K) channels were made from membrane patches excised from CHO cells constitutively expressing the α-subunit of the maxi-K channel or HEK293 cells constitutively expressing both α- and β-subunits using conventional techniques (Hamill et al., 1981, Pflügers Archiv. 391, 85-100) at room temperature. Glass capillary tubing (Garner #7052 or Drummond custom borosilicate glass 1-014-1320) was pulled in two stages to yield micropipettes with tip diameters of approximately 1-2 microns. Pipettes were typically filled with solutions containing (mM): 150 KCl, 10 Hepes (4-(2-hydroxyethyl)-1-piperazine methanesulfonic acid), 1 Mg, 0.01 Ca, and adjusted to pH 7.20 with KOH. After forming a high resistance (>10⁹ ohms) seal between the plasma membrane and the pipette, the pipette was withdrawn from the cell, forming an excised inside-out membrane patch. The patch was excised into a bath solution containing (mM): 150 KCl, 10 Hepes, 5 EGTA (ethylene glycol bis(β-aminoethyl ether)-N,N,N′,N′-tetraacetic acid), sufficient Ca to yield a free Ca concentration of 1-5 μM, and the pH was adjusted to 7.2 with KOH. For example, 4.193 mM Ca was added to give a free concentration of 1 μM at 22° C. An EPC9 amplifier (HEKA Elektronic, Lambrect, Germany) was used to control the voltage and to measure the currents flowing across the membrane patch. The input to the headstage was connected to the pipette solution with a Ag/AgCl wire, and the amplifier ground was connected to the bath solution with a Ag/AgCl wire covered with a tube filled with agar dissolved in 0.2 M KCl. The identity of maxi-K currents was confirmed by the sensitivity of channel open probability to membrane potential and intracellular calcium concentration.

Data acquisition was controlled by PULSE software (HEKA Elektronic) and stored on the hard drive of a MacIntosh computer (Apple Computers) for later analysis using PULSEFIT (HEKA Elektronic) and Igor (Wavemetrics, Oswego, Oreg.) software.

Results:

The effects of the compounds of the present invention on maxi-K channels was examined in excised inside-out membrane patches with constant superfusion of bath solution. The membrane potential was held at −80 mV and brief (100-200 ms) voltage steps to positive membrane potentials (typically +50 mV) were applied once per 15 seconds to transiently open maxi-K channels. As a positive control in each experiment, maxi-K currents were eliminated at pulse potentials after the patch was transiently exposed to a low concentration of calcium (<10 nM) made by adding 1 mM EGTA to the standard bath solution with no added calcium The fraction of channels blocked in each experiment was calculated from the reduction in peak current caused by application of the specified compound to the internal side of the membrane patch. Compound was applied until a steady state level of block was achieved. K_{I} values for channel block were calculated by fitting the fractional block obtained at each compound concentration with a Hill equation. The K_{I} values for channel block by the compounds described in the present invention range from 0.01 nM to greater than 10 μM.

What is claimed is:

1. A compound of the structural formula I: 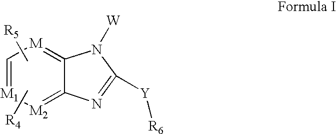
or a pharmaceutically acceptable salt, enantiomer, diastereomer or mixture thereof: wherein,
M, M1, and M2, independently are CH or N;
W represents 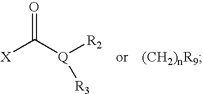
R represents hydrogen, or C₁₋₆ alkyl;
X represents —(CHR₇)ₚ—, or a bond;
Y represents —(CH₂)ᵣ—, —CO(CH₂)ₙ—, —SO₂—, —O—, —S—, —CH(OR′)—, or CONR′;
R′ represents hydrogen, C₁₋₁₀ alkyl, —(CH₂)ₙC₁₋₆ alkoxy, —(CH₂)ₙC₃₋₈ cycloalkyl, —(CH₂)ₙC₃₋₁₀ heterocyclyl, said alkyl, heterocyclyl, aryl or heteroaryl optionally substituted with 1-3 groups selected from R^{a};
or, R′ and R₆ taken together with the intervening N atom of CONR′ of Y to form a 4-10 membered carbocyclic or heterocyclic ring optionally interrupted by 1-3 atoms of O, S, C(O) or NR, and optionally having 1-4 double bonds, and optionally substituted by 1-3 groups selected from R^{a};
Q represents N, CR^{y}, or O, wherein R₂ is absent when Q is O;
R^{y} represents H, C₁₋₁₀ alkyl, C₁₋₆ alkylSR, —(CH₂)ₙO(CH₂)ₘOR,
—(CH₂)ₙC₁₋₆ alkoxy, —(CH₂)ₙC₃₋₈ cycloalkyl, —(CH₂)ₙC₃₋₁₀ heterocyclyl, —(CH₂)ₙC₅₋₁₀ heteroaryl, —N(R)₂, —COOR, or —(CH₂)ₙC₆₋₁₀ aryl, said alkyl, heterocyclyl, aryl or heteroaryl optionally substituted with 1-5 groups selected from R^{a};
or, R₂-Q-R₃ form a 3-15 membered carbocyclic or heterocyclic ring or fused ring, optionally interrupted by 1-3 atoms of O, S, C(O) or NR, and optionally having 1-5 double bonds, and optionally substituted by 1-3 groups selected from R^{a};
R_{w} represents H, C₁₋₆ alkyl, —C(O)C₁₋₆ alkyl, —C(O)OC₁₋₆ alkyl, —SO₂N(R)₂, —SO₂C₁₋₆ alkyl, —SO₂C₆₋₁₀ aryl, NO₂, CN or —C(O)N(R)₂;
R₂ represents hydrogen, C₁₋₁₀ alkyl, C₁₋₆ alkylSR, —(CH₂)ₙO(CH₂)ₘOR, —(CH₂)ₙC₁₋₆ alkoxy, —(CH₂)ₙC₃₋₈ cycloalkyl, —(CH₂)ₙC₃₋₁₀ heterocyclyl, —(CH₂)ₙC₅₋₁₀ heteroaryl, —N(R)₂, —COOR, or —(CH₂)ₙC₆₋₁₀ aryl, said alkyl, heterocyclyl, aryl or heteroaryl optionally substituted with 1-3 groups selected from R^{a};
R₃ represents hydrogen, C₁₋₁₀ alkyl, —(CH₂)ₙC₃₋₈ cycloalkyl, —(CH₂)ₙC₃₋₁₀ heterocyclyl, —(CH₂)ₙC₅₋₁₀ heteroaryl, —(CH₂)ₙCOOR, —(CH₂)ₙC₆₋₁₀ aryl, —(CH₂)ₙNHR₈, —(CH₂)ₙN(R)₂, —(CH₂)ₙNHCOOR, —(CH₂)ₙN(R₈)CO₂R, —(CH₂)ₙN(R₈)COR, —(CH₂)ₙNHCOR, —(CH₂)ₙCONH(R₈), aryl, —(CH₂)ₙC₁₋₆ alkoxy, CF₃, —(CH₂)ₙSO₂R, —(CH₂)ₙSO₂N(R)₂, —(CH₂)ₙCON(R)₂, —(CH₂)ₙCONHC(R)₃, —(CH₂)ₙCOR₈, nitro, cyano or halogen, said alkyl, alkoxy, heterocyclyl, aryl or heteroaryl optionally substituted with 1-3 groups of R^{a};
R₄ and R₅ independently represent hydrogen, C₁₋₆ alkoxy, OH, OCOR₃, C₁₋₆ alkyl, COOR, SO₃H, O(CH₂)ₙN(R)₂, O(CH₂)ₙCO₂R, C₁₋₆ alkylcarbonyl, S(O)_{q}R^{y}, (CH₂)ₙOPO(OH)₂, O(CH₂)ₙOPO(OH)₂, N(R)₂, CF₃, nitro, cyano or halogen where said alkyl, and alkoxy, are optionally substituted with 1-7 groups of R^{a};
R₆ represents hydrogen, C₁₋₁₀ alkyl, —(CH₂)ₙC₆₋₁₀ aryl, —(CH₂)ₙC₅₋₁₀ heteroaryl, (C₆₋₁₀ aryl)O—, —(CH₂)ₙC₃₋₁₀ heterocyclyl, —(CH₂)ₙC₃₋₈ cycloalkyl, —COOR, —C(O)CO₂R, said aryl, heteroaryl, heterocyclyl and alkyl optionally substituted with 1-3 groups selected from R^{a};
R₇ represents hydrogen, C₁₋₆ alkyl, —(CH₂)ₙCOOR or —(CH₂)ₙN(R)₂,
R₈ represents —(CH₂)ₙC₃₋₈ cycloalkyl, —(CH₂)_{n 3-10} heterocyclyl, C₁₋₆ alkoxy or —(CH₂)ₙC₅₋₁₀ heteroaryl, said heterocyclyl, aryl or heteroaryl optionally substituted with 1-3 groups selected from R^{a};
R₉ represents C₁₋₁₀ alkyl, —(CH₂)ₙC₁₋₆ alkoxy, —(CH₂)ₙC₃₋₈ cycloalkyl, —(CH₂)ₙC₃₋₁₀ heterocyclyl, —(CH₂)ₙC₆₋₁₀ aryl, —(CH₂)ₙC₅₋₁₀ heteroaryl, or —N(R)₂ wherein said alkyl, alkoxy, cycloalkyl, heterocyclyl, aryl, or heteroaryl are optionally substituted with 1-3 groups selected from R^{a};
R^{a} represents F, Cl, Br, I, CF₃, N(R)₂, NO₂, CN, —COR₈, —CONHR₈, —CON(R₈)₂, —O(CH₂)ₙCOOR, —NH(CH₂)ₙOR, —COOR, —OCF₃, —NHCOR, —SO₂R, —SO₂NR₂, —SR, (C₁-C₆ alkyl)O—, —(CH₂)ₙO(CH₂)ₘOR, —(CH₂)ₙC₁₋₆ alkoxy, (aryl)O—, —OH, (C₁-C₆ alkyl)S(O)ₘ—, H₂N—C(═NH)—, (C₁-C₆ alkyl)C(O)—, (C₁-C₆ alkyl)OC(O)NH—, —(C₁-C₆ alkyl)NR_{w}(CH₂)ₙC₃₋₁₀ heterocyclyl-R_{w}, —(C₁-C₆ alkyl)O(CH₂)ₙC₃₋₁₀ heterocyclyl-R_{w}, —(C₁-C₆ alkyl)S(CH₂)ₙC₃₋₁₀ heterocyclyl-R_{w}, —(C₁-C₆ alkyl)-C₃₋₁₀ heterocyclyl-R_{w}, —(CH₂)ₙ-Z¹-C(=Z²)N(R)₂, —(C₂₋₆ alkenyl)NR_{w}(CH₂)ₙC₃₋₁₀ heterocyclyl-R_{w}, —(C₂₋₆ alkenyl)O(CH₂)ₙC₃₋₁₀ heterocyclyl-R_{w}, —(C₂₋₆ alkenyl)S(CH₂)ₙC₃₋₁₀ heterocyclyl-R_{w}, —(C₂₋₆ alkenyl)-C₃₋₁₀ heterocyclyl-R_{w}, —(C₂₋₆ alkenyl)-Z¹-C(=Z²)N(R)₂, —(CH₂)ₙSO₂R, —(CH₂)ₙSO₃H, —(CH₂)ₙPO(OR)₂, —(CH₂)ₙOPO(OR)₂, —O(CH₂)ₙSO₂R, —O(CH₂)ₙPO(OR)₂, —O(CH₂)ₙOPO(OR)₂, cyclohexyl, morpholinyl, piperidyl, pyrrolidinyl, thiophenyl, phenyl, pyridyl, imidazolyl, oxazolyl, isoxazolyl, thiazolyl, thienyl, furyl, isothiazolyl, C₂₋₆ alkenyl, and C₁-C₁₀ alkyl, said alkyl, alkenyl, alkoxy, phenyl, pyridyl, imidazolyl, oxazolyl, isoxazolyl, thiazolyl, thienyl, furyl, and isothiazolyl optionally substituted with 1-3 groups selected from C₁-C₆ alkyl, COOR, SO₃H, OH, F, Cl, Br, I, and —O(CH₂)ₙCH(OH)CH₂SO₃H;
Z¹ and Z² independently represents NR_{w}, O, CH₂, or S;
m is 0-3;
n is 0-3;
q is 0-2;
r is 0-6 and
p is 0-2.

2. A compound according to claim 1 wherein M, M1, and M2 are all CH, or at least one of M, M1 or M2 is N.

3. A compound according to claim 2 wherein W represents 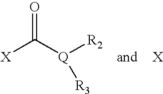
represents CHR₇.

4. A compound according to claim 2 wherein W represents (CH₂)ₙR₉.

5. A compound according to claim 3 wherein Y is —CO(CH₂)ₙ, —(CH₂)ᵣ— or CH(OR) and Q is N or Ry.

6. A compound according to claim 5 wherein R₆ is C₁₋₁₀ alkyl, (CH₂)ₙC₆₋₁₀ aryl, (CH₂)ₙC₅₋₁₀ heteroaryl, (CH₂)ₙC₃₋₁₀ heterocyclyl, or (CH₂)ₙC₃₋₈ cycloalkyl, said aryl, heteroaryl, heterocyclyl and alkyl optionally substituted with 1 to 3 groups of R^{a}, Y is —CO(CH₂)ₙ, Q is N, and R₂ and R₃ are independently selected from C₁₋₁₀ alkyl, (CH₂)ₙC₃₋₈ cycloalkyl, —(CH₂)ₙ-5˜10-membered heteroaryl, —(CH₂)ₙC₆₋₁₀ aryl, —(CH₂)ₙ-3˜10-membered heterocyclyl, and C₁₋₆ akylOH said cycloalkyl, aryl, heteroaryl, heterocyclyl and alkyl optionally substituted with 1 to 3 groups of R^{a}.

7. A compound which is:
1-(1-Benzyl-6-methoxy-1H-benzimidazol-2-yl)-2,2-dimethylpropan-1-one,
1-(1-benzyl-5-methoxy-1H-benzimidazol-2-yl)-2,2-dimethylpropan-1-one,
1-(5-Methoxy-1H-benzimidazol-2-yl)-2,2-dimethylpropan-1-one,
Methyl[2-(2,2-dimethylpropanoyl)-6-methoxy-1H-benzimidazol-1-yl]acetate,
Methyl[2-(2,2-dimethylpropanoyl)-5-methoxy-1H-benzimidazol-1-yl]acetate,
[2-(2,2-Dimethylpropanoyl)-5-methoxy-1H-benzimidazol-1-yl]acetic acid,
2-[2-(2,2-Dimethylpropanoyl)-5-methoxy-1H-benzimidazol-1-yl]-N,N-bis(3-methylbutyl)acetamide,
1-(Diethoxymethyl)-6-methoxy-1H-benzimidazole,
1-(diethoxymethyl)-5-methoxy-1H-benzimidazole,
1-(6-Methoxy-1H-benzimidazol-2-yl)-2,2-dimethylpropan-1-one,
N,N-Dibutyl-2-[2-(2,2-dimethylpropanoyl)-5-methoxy-1H-benzimidazol-1-yl]acetamide,
2-[2-2,2-Dimethylpropanoyl)-5-methoxy-1H-benzimidazol-1-yl]-N,N-diisobutylacetamide,
2-[2-(2,2-Dimethylpropanoyl)-5-methoxy-1H-benzimidazol-1-yl]-N,N-dipropylacetamide,
N-(Cyclopropylmethyl)-2-[2-2,2-dimethylpropanoyl)-5-methoxy-1H-benzimidazol 1-yl]-N-propylacetamide,
2-[2-(2,2-Dimethylpropanoyl)-5-methoxy-1H-benzimidazol-1-yl]-N-ethyl-N-(3-methylbutyl)acetamide,
N-Butyl-2-[2-(2,2-dimethylpropanoyl)-5-methoxy-1H-benzimidazol-1-yl]-N-ethylacetamide,
N-Cyclohexyl-2-[2-(2,2-dimethylpropanoyl)-5-methoxy-1H-benzimidazol-1-yl]-N-ethylacetamide,
2-[2-(2,2-Dimethylpropanoyl)-5-methoxy-1H-benzimidazol-1-yl]-N-ethyl-N-1,3-thiazol-2-ylacetamide,
[2-(2,2-Dimethylpropanoyl)-6-methoxy-1H-benzimidazol-1-yl]acetic acid,
2-[2-(2,2-Dimethylpropanoyl)-6-methoxy-1H-benzimidazol-1-yl]-N,N-bis(3-methylbutyl)acetamide,
N,N-Dibutyl-2-[2-(2,2-dimethylpropanoyl)-6-methoxy-1H-benzimidazol-1-yl]acetamide,
2-[2-(2,2-Dimethylpropanoyl)-6-methoxy-1H-benzimidazol-1-yl]-N,N-diisobutylacetamide,
2-[2-(2,2-Dimethylpropanoyl)-6-methoxy-1H-benzimidazol-1-yl]-N,N-dipropylacetamide,
N-(Cyclopropylmethyl)-2-[2-(2,2-dimethylpropanoyl)-6-methoxy-1H-benzimidazol-1-yl]-N-propylacetamide,
2-[2-(2,2-Dimethylpropanoyl)-6-methoxy-1H-benzimidazol-1-yl]-N-ethyl-N-(3-methylbutyl)acetamide,
N-Butyl-2-[2-(2,2-dimethylpropanoyl)-6-methoxy-1H-benzimidazol-1-yl]-N-ethylacetamide,
N-Cyclohexyl-2-[2-(2,2-dimethylpropanoyl)-6-methoxy-1H-benzimidazol-1-yl]-N-ethylacetamide,
2-[2-(2,2-Dimethylpropanoyl)-6-methoxy-1H-benzimidazol-1-yl]-N-ethyl-N-1,3-thiazol-2-ylacetamide,
N-(3,3-Dimethylbutyl)-2-[2-2,2-dimethylpropanoyl)-6-methoxy-1H-benzimidazol-1-yl]-N-ethylacetamide,
1-[2-(2,2-Dimethylpropanoyl)-5-methoxy-1H-benzimidazol-1-yl]-3,3-dimethylbutan-2-one, 1-[2-(2,2-Dimethylpropanoyl)-6-methoxy-1H-benzimidazol-1-yl]-3,3-dimethylbutan-2-one,
1-(1-Benzyl-5-methoxy-1H-benzimidazol-2-yl)-2,2-dimethylpropan-1-one,
1-(1-Benzyl-6-methoxy-1H-benzimidazol-2-yl)-2,2-dimethylpropan-1-one,
1-[1-(3,3-Dimethylbutyl)-5-methoxy-1H-benzimidazol-2-yl]-2,2-dimethylpropan-1-one,
1-[1-(3,3-Dimethylbutyl)-6-methoxy-1H-benzimidazol-2-yl]-2,2-dimethylpropan-1-one,
N,N-Dibutyl-2-[2-(2,2-dimethylpropyl)-5-methoxy-1H-benzimidazol-1-yl]acetamide,
N,N-Dibutyl-2-[2-(2,2-dimethylpropyl)-6-methoxy-1H-benzimidazol-1-yl]acetamide,
1-[2-(2,2-Dimethylpropyl)-5-methoxy-1H-benzimidazol-1-yl]-3,3-dimethylbutan-2-one,
1-[2-(2,2-Dimethylpropyl)-6-methoxy-1H-benzimidazol-1-yl]-3,3-dimethylbutan-2-one,
1-[5-Methoxy-2-(2-phenylethyl)-1H-benzimidazol-1-yl]-3,3-dimethylbutan-2-one,
1-[6-Methoxy-2-(2-phenylethyl)-1H-benzimidazol-1-yl]-3,3-dimethylbutan-2-one,
1-(5-Methoxy-2-phenyl-1H-benzimidazol-1-yl)-3,3-dimethylbutan-2-one,
1-(6-Methoxy-2-phenyl-1H-benzimidazol-1-yl)-3,3-dimethylbutan-2-one,
1-(2-Benzyl-5-methoxy-1H-benzimidazol-1-yl)-3,3-dimethylbutan-2-one,
1-(2-Benzyl-6-methoxy-1H-benzimidazol-1-yl)-3,3-dimethylbutan-2-one,
N,N-dibutyl-2-(2-isobutyryl-6-methoxy-1H-imidazo[4,5-c]pyridin-1-yl)acetamide,
N,N-dibutyl-2-(2-isobutyryl-5-methoxy-3H-imidazo[4,5-b]pyridin-3-yl)acetamide,
N,N-dibutyl-2-(2-isobutyryl-6-methoxy-1H-imidazo[4,5-b]pyridin-1-yl)acetamide,
N,N-dibutyl-2-(8-isobutyryl-2-methoxy-9H-purin-9-yl)acetamide,
N,N-dibutyl-2-(2-isobutyryl-6-methoxy-1H-imidazo[4,5-b]pyrazin-1-yl)acetamide,
N,N-dibutyl-2-(6-isobutyryl-3-methoxy-5H-imidazo[4,5-c]pyridazin-5-yl)acetamide,
N,N-dibutyl-2-(6-isobutyryl-3-methoxy-5H-imidazo[4,5-e][1,2,4]triazin-5-yl)acetamide,
N,N-dibutyl-2-[2-(2,2-dimethylpropanoyl)-5-methoxy-3H-imidazo[4,5-b]pyridin-3-yl]acetamide
N,N-dibutyl-2-(2-(2,2-dimethylpropanoyl)-6-methoxy-1H-imidazo[4,5-c]pyridin-1-yl)acetamide,
N,N-dibutyl-2-(2-(2,2-dimethylpropanoyl)-6-methoxy-1H-imidazo[4,5-b]pyridin-1-yl)acetamide,
N,N-dibutyl-2-(8-(2,2-dimethylpropanoyl)-2-methoxy-9H-purin-9-yl)acetamide,
N,N-dibutyl-2-(2-(2,2-dimethylpropanoyl)-6-methoxy-1H-imidazo[4,5-b]pyrazin-1-yl)acetamide,
N,N-dibutyl-2-(6-(2,2-dimethylpropanoyl)-3-methoxy-5H-imidazo[4,5-c]pyridazin-5-yl)acetamide,
N,N-dibutyl-2-(6-(2,2-dimethylpropanoyl)-3-methoxy-5H-imidazo[4,5-e][1,2,4]triazin-5-yl)acetamide,
2-[2-(2,2-dimethylpropanoyl)-5-methoxy-3H-imidazo[4,5-b]pyridin-3-yl]-N,N-bis(3-methylbutyl)acetamide,
2-(2-(2,2-dimethylpropanoyl)-6-methoxy-1H-imidazo[4,5-c]pyridin-1-yl)-N,N-bis(3-methylbutyl)acetamide,
2-(2-(2,2-dimethylpropanoyl)-6-methoxy-1H-imidazo[4,5-b]pyridin-1-yl)-N,N-bis(3-methylbutyl)acetamide,
2-(8-(2,2-dimethylpropanoyl)-2-methoxy-9H-purin-9-yl)-N,N-bis(3-methylbutyl)acetamide,
2-(2-(2,2-dimethylpropanoyl)-6-methoxy-1H-imidazo[4,5-b]pyrazin-1-yl)-N,N-bis(3-methylbutyl)acetamide,
2-(6-(2,2-dimethylpropanoyl)-3-methoxy-5H-imidazo[4,5-c]pyridazin-5-yl)-N,N-bis(3-methylbutyl)acetamide,
2-[6-(2,2-dimethylpropanoyl)-3-methoxy-5H-imidazo[4,5-e][1,2,4]triazin-5-yl]-N,N-bis(3-methylbutyl)acetamide,
2-(2-isobutyryl-5-methoxy-3H-imidazo[4,5-b]pyridin-3-yl)-N,N-bis(3-methylbutyl)acetamide,
2-2 isobutyryl-6-methoxy-1H-imidazo[4,5-c]pyridin-1-yl)-N,N-bis(3-methylbutyl)acetamide,
2-(2-isobutyryl-6-methoxy-1H-imidazo[4,5-b]pyridin-1-yl)-N,N-bis(3-methylbutyl)acetamide,
2-(8-isobutyryl-2-methoxy-9H-purin-9-yl)-N,N-bis(3-methylbutyl)acetamide,
2-(2-isobutyryl-6-methoxy-1H-imidazo[4,5-b]pyrazin-1-yl)-N,N-bis(3-methylbutyl)acetamide,
2-(6-isobutyryl-3-methoxy-5H-imidazo[4,5-c]pyridazin-5-yl)-N,N-bis(3-methylbutyl)acetamide,
2-[6-(2,2-dimethylpropanoyl)-3-methoxy-5H-imidazo[4,5-e][1,2,4]triazin-5-yl]-N,N-bis(3-methylbutyl)acetamide,
1-(2-benzoyl-6-methoxy-1H-benzimidazol-1-yl)-3,3-dimethylbutan-2-one,
2-(2-benzoyl-6-methoxy-1H-benzimidazol-1-yl)-N,N-dibutylacetamide,
2-(2-benzoyl-6-methoxy-1H-benzimidazol-1-yl)-N,N-bis(3-methylbutyl)acetamide,
2-(2-benzoyl-6-methoxy-1H-benzimidazol-1-yl)-N-butyl-N-ethylacetamide,
2-(2-benzoyl-6-methoxy-1H-benzimidazol-1-yl)-N,N-dipropylacetamide,
2-(2-benzoyl-6-methoxy-1H-benzimidazol-1-yl)-N-(tert-butyl)-N-ethylacetamide,
2-(2-benzoyl-6-methoxy-1H-benzimidazol-1-yl)-N-ethyl-N-1,3-thiazol-2-ylacetamide,
[6-methoxy-1-(3-methylbutyl)-1H-benzimidazol-2-yl](phenyl)methanone,
[1-(2-ethylbutyl)-6-methoxy-1H-benzimidazol-2-yl](phenyl)methanone,
[1-(3,3-dimethylbutyl)-6-methoxy-1H-benzimidazol-2-yl](phenyl)methanone,
N-benzyl-2-[2-(2,2-dimethylpropanoyl)-6-methoxy-1H-benzimidazol-1-yl]-N-ethylacetamide,
2-(2-isobutyryl-6-methoxy-1H-benzimidazol-1-yl)-N,N-bis(3-methylbutyl)acetamide,
N,N-dibutyl-2-(2-isobutyryl-6-methoxy-1H-benzimidazol-1-yl)acetamide,
N,N-diisobutyl-2-(2-isobutyryl-6-methoxy-H-benzimidazol-1-yl)acetamide,
2-(2-isobutyryl-6-methoxy-1H-benzimidazol-1-yl)-N,N-dipropylacetamide,
N-(cyclopropylmethyl)-2-(2-isobutyryl-6-methoxy-1H-benzimidazol-1-yl)-N-propylacetamide,
N-ethyl-2-(2-isobutyryl-6-methoxy-1H-benzimidazol-1-yl)-N-(3-methylbutyl)acetamide,
N-butyl-N-ethyl-2-(2-isobutyryl-6-methoxy-1H-benzimidazol-1-yl)acetamide,
N-cyclohexyl-N-ethyl-2-(2-isobutyryl-6-methoxy-1H-benzimidazol-1-yl)acetamide,
N-butyl-2-[2-2,2-dimethylpropanoyl)-6-methoxy-1H-benzimidazol-1-yl]-N-propylacetamide,
1-(1-{2-[trans-2,5-dipropylpyrrolidin-1-yl]-2-oxoethyl}-6-methoxy-1H-benzimidazol-2-yl)-2,2-dimethylpropan-1-one,
1-(1-{2-[cis-2,5-dipropylpyrrolidin-1-yl]-2-oxoethyl}-6-methoxy-1H-benzimidazol-2-yl)-2,2-dimethylpropan-1-one,
1-(2-isobutyryl-6-methoxy-1H-benzimidazol-1-yl)-3,3-dimethylbutan-2-one,
N-(3,3-dimethylbutyl)-N-ethyl-2-(2-isobutyryl-6-methoxy-1H-benzimidazol-1-yl)acetamide,
N-butyl-2-(2-isobutyryl-6-methoxy-1H-benzimidazol-1-yl)-N-propylacetamide,
N-(3,3-dimethylbutyl)-2-[2-(2,2-dimethylpropanoyl)-6-methoxy-1H-benzimidazol-1-yl]-N-propylacetamide,
2-[2-(2,2-dimethylpropanoyl)-6-methoxy-1H-benzimidazol-1-yl]-N-(2,2-dimethylpropyl)-N-ethylacetamide,
2-{2-[4-(hydroxymethyl)benzoyl]-6-methoxy-1H-benzimidazol-1-yl}-N,N-bis(3-methylbutyl)acetamide,
2-{2-[4-(hydroxymethyl)benzoyl]-6-methoxy-1H-benzimidazol-1-yl}-N,N-diisobutylacetamide,
N-(3,3-dimethylbutyl)-N-ethyl-2-{2-[4-(hydroxymethyl)benzoyl]-6-methoxy-1H-benzimidazol-1-ylacetamide,
2-{2-[(4-trans-hydroxycyclohexyl)carbonyl]-6-methoxy-1H-benzimidazol-1-yl}-N,N-bis(3-methylbutyl)acetamide,
N-(3,3-dimethylbutyl)-2-{2-[(4-trans-hydroxycyclohexyl)carbonyl]-6-methoxy-1H-benzimidazol-1-yl}-N-propylacetamide,
N-(3,3-dimethylbutyl)-N-ethyl-2-{2-[(4-trans-hydroxycyclohexyl)carbonyl]-6-methoxy-1H-benzimidazol-1-yl}acetamide,
N,N-bis(3,3-dimethylbutyl)-2-(2-[(4-trans-hydroxycyclohexyl)carbonyl]-6-methoxy-1H-benzimidazol-1-yl}acetamide,
2-{2-[(4-cis-hydroxycyclohexyl)carbonyl]-6-methoxy-1H-benzimidazol-1-yl}-N,N-bis(3-methylbutyl)acetamide,
2-(2-{[4-(hydroxymethyl)-1-methylcyclohexyl]carbonyl}-6-methoxy-1H-benzimidazol-1-yl)-N,N-bis(3-methylbutyl)acetamide,
N,N-dibutyl-2-(2-{[4-(hydroxymethyl)-1-methylcyclohexyl]carbonyl}-6-methoxy-1H-benzimidazol-1-yl)acetamide,
2-(2-{[4-(hydroxymethyl)-1-methylcyclohexyl]carbonyl}-6-methoxy-1H-benzimidazol-1-yl)-N,N-diisobutylacetamide,
N-(3,3-dimethylbutyl)-N-ethyl-2-(2-{[4-(hydroxymethyl)-1-methylcyclohexyl]carbonyl}-6-methoxy-1H-benzimidazol-1-yl)acetamide,
N-butyl-2-(2-{[4-(hydroxymethyl)-1-methylcyclohexyl]carbonyl}-6-methoxy-1H-benzimidazol-1-yl)-N-propylacetamide,
N-(3,3-dimethylbutyl)-2-(2-{[4-(hydroxymethyl)-1-methylcyclohexyl]carbonyl}-6-methoxy-1H-benzimidazol-1-yl)-N-propylacetamide,
N-ethyl-2-(2-{[4-(hydroxymethyl)-1-methylcyclohexyl]carbonyl}-6-methoxy-1H-benzimidazol-1-yl)-N-(3-methylbutyl)acetamide,
1-{1-[2-(1-adamantyl)-2-oxoethyl)-6-methoxy-1H-benzimidazol-2-yl}-2,2-dimethylpropan-1-one,
1-{1-[2-(1-adamantyl)-2-oxoethyl]-6-methoxy-1H-benzimidazol-2-yl}-2-methylpropan-1-one,
1-(2-benzyl-5-methoxy-1H-benzimidazol-1-yl)-3,3-dimethylbutan-2-one,
1-(5-methoxy-2-phenyl-1H-benzimidazol-1-yl)-3,3-dimethylbutan-2-one,
1-(5-methoxy-2-(2-phenylethyl)-1H-benzimidazol-1-yl]-3,3-dimethylbutan-2-one,
or a pharmaceutically acceptable salt, enantiomer, diastereomer or mixture thereof.

8. A method for treating ocular hypertension or glaucoma comprising administration to a patient in need of such treatment a therapeutically effective amount of a compound of structural formula I of claim 1.

9. A method for treating macular edema, macular degeneration, increasing retinal and optic nerve head blood velocity, increasing retinal and optic nerve oxygen tension, and/or a neuroprotective effect comprising administration to a patient in need of such treatment a pharmaceutically effective amount of a compound of claim 1; or a pharmaceutically acceptable salt, enantiomer, diastereomer or mixture thereof.

10. A method of preventing repolarization or hyperpolarization of a mammalian cell containing potassium channel or a method of treating Alzheimer's Disease, depression, cognitive disorders, and/or arrhythmia disorders in a patient in need thereof comprising administering a pharmaceutically effective amount of a compound according to claim 1, or a pharmaceutically acceptable salt, enantiomer, diastereomer or mixture thereof.

11. A method of treating diabetes in a patient in need thereof comprising administering a pharmaceutically effective amount of a compound according to claim 1, or a pharmaceutically acceptable salt, enantiomer, diastereomer or mixture thereof.

12. A composition comprising a compound of formula I of claim 1 and a pharmaceutically acceptable carrier.

13. The composition according to claim 12 wherein the compound of formula I is applied as a topical formulation, said topical formulation administered as a solution or suspension and optionally containing xanthan gum or gellan gum.

14. A composition according to claim 13 wherein one or more of an active ingredient belonging to the group consisting of: β-adrenergic blocking agent, parasympatho-mimetic agent, sympathomimetic agent, carbonic anhydrase inhibitor, EP4 agonist, a prostaglandin or derivative thereof, hypotensive lipid, neuroprotectant, and/or 5-HT2 receptor agonist is optionally added.

15. A composition according to claim 14 wherein the β-adrenergic blocking agent is timolol, betaxolol, levobetaxolol, carteolol, or levobunolol; the parasympathomimetic agent is pilocarpine; the sympathomimetic agent is epinephrine, brimonidine, iopidine, clonidine, or para-aminoclonidine, the carbonic anhydrase inhibitor is dorzolamide, acetazolamide, metazolamide or brinzolamide; the prostaglandin is latanoprost, travaprost, unoprostone, rescula, or S1033, the hypotensive lipid is lumigan, the neuroprotectant is eliprodil, R-eliprodil or memantine; and the 5-HT2 receptor agonist is 1-(2-aminopropyl)-3-methyl-1H-imdazol-6-ol fumarate or 2-(3-chloro-6-methoxy-indazol-1-yl)-1-methyl-ethylamine.
